SEQUENCE LISTING (117 sequences)

SEQ ID NO: 1 - protein, 7 aa, Synthetic construct
TVRTSAD

SEQ ID NO: 2 - protein, 7 aa, synthetic construct
VTRTSAD

SEQ ID NO: 3 - protein, 7 aa, synthetic construct
AVRTSAD

SEQ ID NO: 4 - protein, 7 aa, synthetic construct
TARTSAD

SEQ ID NO: 5 - protein, 7 aa, synthetic construct
TVATSAD

SEQ ID NO: 6 - protein, 7 aa, synthetic construct
TVRASAD

SEQ ID NO: 7 - protein, 7 aa, synthetic construct
TVRTSAA

SEQ ID NO: 8 - protein, 7 aa, synthetic construct
HVRTSAD

SEQ ID NO: 9 - protein, 7 aa, synthetic construct
THRTSAD

SEQ ID NO: 10 - protein, 7 aa, synthetic construct
TVHTSAD

SEQ ID NO: 11 - protein, 7 aa, synthetic construct
TVRHSAD

SEQ ID NO: 12 - protein, 7 aa, synthetic construct
TVRTSAH

SEQ ID NO: 13 - protein, 7 aa, synthetic construct
HVRTSAD

SEQ ID NO: 14 - protein, 7 aa, synthetic construct
AHRTSAD

SEQ ID NO: 15 - protein, 7 aa, synthetic construct
AVHTSAD

SEQ ID NO: 16 - protein, 7 aa, synthetic construct
AVRHSAD

SEQ ID NO: 17 - protein, 7 aa, synthetic construct
AVRTHAD

SEQ ID NO: 18 - protein, 7 aa, synthetic construct
AVRTSAH

SEQ ID NO: 19 - protein, 7 aa, synthetic construct
AARTSAD

SEQ ID NO: 20 - protein, 7 aa, synthetic construct
AVATSAD

SEQ ID NO: 21 - protein, 7 aa, synthetic construct
AVRASAD

SEQ ID NO: 22 - protein, 7 aa, synthetic construct
AVRTAAD

SEQ ID NO: 23 - protein, 7 aa, synthetic construct
AVRTSAA

SEQ ID NO: 24 - protein, 7 aa, synthetic construct
NVRTSAD

SEQ ID NO: 25 - protein, 7 aa, synthetic construct
TNRTSAD

SEQ ID NO: 26 - protein, 7 aa, synthetic construct
TVNTSAD

SEQ ID NO: 27 - protein, 7 aa, synthetic construct
TVRNSAD

SEQ ID NO: 28 - protein, 7 aa, synthetic construct
TVRTSAN

SEQ ID NO: 29 - protein, 7 aa, synthetic construct
NVRTSAD

SEQ ID NO: 30 - protein, 7 aa, synthetic construct
ANRTSAD

SEQ ID NO: 31 - protein, 7 aa, synthetic construct
AVNTSAD

SEQ ID NO: 32 - protein, 7 aa, synthetic construct
AVRNSAD

SEQ ID NO: 33 - protein, 7 aa, synthetic construct
AVRTNAD

SEQ ID NO: 34 - protein, 7 aa, synthetic construct
AVRTSAN

SEQ ID NO: 35 - protein, 7 aa, synthetic construct
  SITE 2: note = D-Valine
TVRTSAD

SEQ ID NO: 36 - protein, 7 aa, synthetic construct
  SITE 3: note = D-Arginine
TVRTSAD

SEQ ID NO: 37 - protein, 7 aa, synthetic construct
  SITE 4: note = D-Threonine
TVRTSAD

SEQ ID NO: 38 - protein, 7 aa, synthetic construct
  SITE 5: note = D-Serine
TVRTSAD

SEQ ID NO: 39 - protein, 7 aa, synthetic construct
  SITE 6: note = D-Alanine
TVRTSAD

SEQ ID NO: 40 - protein, 7 aa, synthetic construct
  SITE 7: note = D-Aspartic acid (Aspartate)
TVRTSAD

SEQ ID NO: 41 - protein, 7 aa, synthetic construct
  SITE 2: note = D-Valine
AVRTSAD

SEQ ID NO: 42 - protein, 7 aa, synthetic construct
  SITE 3: note = D-Arginine
AVRTSAD

SEQ ID NO: 43 - protein, 7 aa, synthetic construct
  SITE 7: note = D-Aspartic acid (Aspartate)
AVRTSAD

SEQ ID NO: 44 - protein, 7 aa, synthetic construct
  SITE 3: note = D-Arginine
AVRTSAH

SEQ ID NO: 45 - protein, 6 aa, synthetic construct
TVRTSA

SEQ ID NO: 46 - protein, 6 aa, synthetic construct
AVRTSA

SEQ ID NO: 47 - protein, 6 aa, synthetic construct
VRTSAD

SEQ ID NO: 48 - protein, 7 aa, synthetic construct
NWGDRIL

SEQ ID NO: 49 - protein, 7 aa, synthetic construct
AWGDRIL

SEQ ID NO: 50 - protein, 7 aa, synthetic construct
NAGDRIL

SEQ ID NO: 51 - protein, 7 aa, synthetic construct
NWADRIL

SEQ ID NO: 52 - protein, 7 aa, synthetic construct
NWGARIL

SEQ ID NO: 53 - protein, 7 aa, synthetic construct
NWGDAIL

SEQ ID NO: 54 - protein, 7 aa, synthetic construct
NWGDRAL

SEQ ID NO: 55 - protein, 7 aa, synthetic construct
NWGDRIA

SEQ ID NO: 56 - protein, 7 aa, synthetic construct
NWGKPIK

SEQ ID NO: 57 - protein, 7 aa, synthetic construct
AWGKPIK

SEQ ID NO: 58 - protein, 7 aa, synthetic construct
NAGKPIK

SEQ ID NO: 59 - protein, 7 aa, synthetic construct
NWAKPIK

SEQ ID NO: 60 - protein, 7 aa, synthetic construct
NWGAPIK

SEQ ID NO: 61 - protein, 7 aa, synthetic construct
NWGKAIK

SEQ ID NO: 62 - protein, 7 aa, synthetic construct
NWGKPAK

SEQ ID NO: 63 - protein, 7 aa, synthetic construct
NWGKPIA

SEQ ID NO: 64 - protein, 7 aa, synthetic construct
SGVKSAF

SEQ ID NO: 65 - protein, 7 aa, synthetic construct
AGVKSAF

SEQ ID NO: 66 - protein, 7 aa, synthetic construct
SAVKSAF

SEQ ID NO: 67 - protein, 7 aa, synthetic construct
SGAKSAF

SEQ ID NO: 68 - protein, 7 aa, synthetic construct
SGVASAF

SEQ ID NO: 69 - protein, 7 aa, synthetic construct
SGVKAAF

SEQ ID NO: 70 - protein, 7 aa, synthetic construct
SGVKSAA

SEQ ID NO: 71 - protein, 7 aa, synthetic construct
GVKSYNE

SEQ ID NO: 72 - protein, 7 aa, synthetic construct
AVKSYNE

SEQ ID NO: 73 - protein, 7 aa, synthetic construct
GAKSYNE

SEQ ID NO: 74 - protein, 7 aa, synthetic construct
GVASYNE

SEQ ID NO: 75 - protein, 7 aa, synthetic construct
GVKAYNE

SEQ ID NO: 76 - protein, 7 aa, synthetic construct
GVKSANE

SEQ ID NO: 77 - protein, 7 aa, synthetic construct
GVKSYAE

SEQ ID NO: 78 - protein, 7 aa, synthetic construct
GVKSYNA

SEQ ID NO: 79 - protein, 7 aa, synthetic construct
IGKTNTL

SEQ ID NO: 80 - protein, 7 aa, synthetic construct
AGKTNTL

SEQ ID NO: 81 - protein, 7 aa, synthetic construct
IAKTNTL

SEQ ID NO: 82 - protein, 7 aa, synthetic construct
IGATNTL

SEQ ID NO: 83 - protein, 7 aa, synthetic construct
IGKANTL

SEQ ID NO: 84 - protein, 7 aa, synthetic construct
IGKTATL

SEQ ID NO: 85 - protein, 7 aa, synthetic construct
IGKTNAL

SEQ ID NO: 86 - protein, 7 aa, synthetic construct
IGKTNAA

SEQ ID NO: 87 - protein, 7 aa, synthetic construct
IGNSNTL

SEQ ID NO: 88 - protein, 7 aa, synthetic construct
AGNSNTL

SEQ ID NO: 89 - protein, 7 aa, synthetic construct
IANSNTL

SEQ ID NO: 90 - protein, 7 aa, synthetic construct
IGASNTL

SEQ ID NO: 91 - protein, 7 aa, synthetic construct
IGNANTL

SEQ ID NO: 92 - protein, 7 aa, synthetic construct
IGNSATL

SEQ ID NO: 93 - protein, 7 aa, synthetic construct
IGNSNAL

SEQ ID NO: 94 - protein, 7 aa, synthetic construct
IGNSNTA

SEQ ID NO: 95 - protein, 7 aa, synthetic construct
IGNTIPV

SEQ ID NO: 96 - protein, 7 aa, synthetic construct
AGNTIPV

SEQ ID NO: 97 - protein, 7 aa, synthetic construct
IANTIPV

SEQ ID NO: 98 - protein, 7 aa, synthetic construct
IGATIPV

SEQ ID NO: 99 - protein, 7 aa, synthetic construct
IGNAIPV

SEQ ID NO: 100 - protein, 7 aa, synthetic construct
IGNTAPV

SEQ ID NO: 101 - protein, 7 aa, synthetic construct
IGNTIAV

SEQ ID NO: 102 - protein, 7 aa, synthetic construct
IGNTIPA

SEQ ID NO: 103 - protein, 7 aa, synthetic construct
LYANSPF

SEQ ID NO: 104 - protein, 7 aa, synthetic construct
AYANSPF

SEQ ID NO: 105 - protein, 7 aa, synthetic construct
LAANSPF

SEQ ID NO: 106 - protein, 7 aa, synthetic construct
LYAASPF

SEQ ID NO: 107 - protein, 7 aa, synthetic construct
LYANAPF

SEQ ID NO: 108 - protein, 7 aa, synthetic construct
LYANSAF

SEQ ID NO: 109 - protein, 7 aa, synthetic construct
LYANSPA

SEQ ID NO: 110 - protein, 7 aa, synthetic construct
  SITE 3: note = D-Arginine
AVRTSAN

SEQ ID NO: 111 - protein, 6 aa, synthetic construct
ARTSAD

SEQ ID NO: 112 - protein, 7 aa, synthetic construct
TLRTSAD

SEQ ID NO: 113 - protein, 7 aa, synthetic construct
ALRTSAH

SEQ ID NO: 114 - protein, 7 aa, synthetic construct
TLRTSAT

SEQ ID NO: 115 - protein, 7 aa, synthetic construct
ALRTSAT

SEQ ID NO: 116 - protein, 7 aa, synthetic construct
TLRTSAS

SEQ ID NO: 117 - protein, 7 aa, synthetic construct
ALRTSAS

(19) United States
(12) Patent Application Publication
(10) Pub. No.: US 2024/0325574 A1
(43) Pub. Date: Oct. 3, 2024

(54) EXTRADOMAIN B FIBRONECTIN TARGETING PEPTIDES AND DERIVATIVES FOR CANCER IMAGING AND THERAPY

(71) Applicant: MOLECULAR THERANOSTICS, LLC, Cleveland, OH (US)

(72) Inventors: Songqui Gao, Beachwood, OH (US); Michael Tweedle, Bexley, OH (US); Yajuan Li, Princeton Junction, NJ (US); Zheng-Rong Lu, Beachwood, OH (US)

(21) Appl. No.: 18/625,695

(22) Filed: Apr. 3, 2024

Related U.S. Application Data
(60) Provisional application No. 63/456,687, filed on Apr. 3, 2023.

(51) Int. Cl. A61K 49/10; A61K 47/62; A61K 49/00

(52) U.S. Cl. CPC A61K 49/106; A61K 47/62; A61K 49/0032; A61K 2123/00

(57) ABSTRACT

Molecular probe compounds according to the formula P-L-I are described. P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide. Methods of detecting cancer and monitoring the treatment of cancer using the molecular probes are also described.

### CROSS-REFERENCE TO RELATED APPLICATIONS

This application claims priority to U.S. Provisional Application No. 63/456,687, filed on Apr. 3, 2023, which is incorporated herein by reference.

### GOVERNMENT FUNDING

The present invention was made with government support under Grant No. R44CA265626 awarded by the National Institutes of Health. The US government has certain rights in this invention.

### SEQUENCE LISTING

The instant application contains a Sequence Listing XML (Name: MOL-032636 US ORD-SeqListing-ST26.xml; size: 105,932 bytes; and Date of Creation: Apr. 2, 2024) which has been submitted in XML format via EFS-Web and is hereby incorporated by reference in its entirety.

### BACKGROUND

Cancer detection and treatment are hindered by the inability to differentiate between cancer cells and normal cells. Better detection tools for cancer or tumor imaging are needed for earlier diagnosis of cancers. Molecular recognition of tumor cells would facilitate guided surgical resection. In order to improve surgical resection, targeted imaging tools must specifically label tumor cells, not only in the main tumor but also along the edge of the tumor and in the small tumor cell clusters that disperse throughout the body. Targeted imaging tools designed to label molecules that accumulate in the tumor microenvironment may also be advantageous as therapeutic targeting agents, as they can identify both the main tumor cell population and areas with infiltrating cells that contribute to tumor recurrence. The ability to directly target the tumor cell and/or its microenvironment would increase both the specificity and sensitivity of current treatments, therefore reducing non-specific side effects of chemotherapeutics that affect cells throughout the body.

The inventors believe that design and development of small molecular targeted agents specific to abundant oncoproteins in tumor extracellular matrix (ECM) would overcome the current limitations of cancer molecular imaging and precision cancer diagnosis and therapy. Tumors have a unique microenvironment enriched with oncogenic ECM proteins, not present in normal tissues. Among various ECM proteins in tumor tissues, extradomain B fibronectin (EDB-FN) is a promising target for designing clinically translatable targeted GBCA. EDB-FN is an oncofetal isoform of fibronectin (FN) and is highly expressed in the ECM of aggressive tumors.

EDB-FN is a marker of Epithelial Mesenchymal Transition (EMT), which is a biological program associated with invasion, metastasis, and drug resistance of multiple malignancies. Elevated expression of EDB-FN is associated with EMT induction, cancer cell invasion, and metastasis. It is overexpressed in various aggressive human cancers and absent in normal tissues. EDB-FN has been explored as an oncotarget for developing molecular imaging technologies and targeted therapeutics for cancer diagnosis and therapy. For example, antibody fragment L19 specific to EDB-FN has been used to develop positron emission tomography (PET) probes and targeted therapeutics for cancer imaging and therapy in both preclinical and clinical studies. A nanobody has been developed to target EDB-FN for effective PET imaging of different types of cancer in animal tumor models. An aptamer-like peptide has also been developed to target EDB-FN to deliver imaging agents and therapeutics for cancer imaging and therapy.

To encourage clinically and industrially acceptable properties, the inventors have used phage display against EDB and developed 20 small peptides, as described in U.S. Pat. No. 10,653,801. Among them, a peptide named ZD2 (Thr-Val-Arg-Thr-Ser-Ala-Asp; SEQ ID NO: 1) that has a moderate binding affinity to the EDB fragment has been chosen for further development. The peptide was conjugated to a clinical contrast agent Gd (HP-DO3A), making ZD2-Gd (HP-DO3A). The agent was effective for MRMI of aggressive tumor detection and characterization of aggressive prostate cancer and metastatic breast cancer in mouse models. The structure of the targeted contrast agent was further optimized to identify a lead targeted contrast agent ZD2-N3-Gd (HP-DO3A), or MT218 with improved T1 relaxivity for clinical translation. MT218 produces robust specific MR contrast enhancement at doses lower than those of the base GBCA in several types of aggressive tumors, including breast cancer, colorectal cancer, head and neck cancer, pancreatic cancer, and prostate cancer. The enhancement pattern closely reflects relative EDB-FN expression levels in these tissue types, which is known to reflect tumor aggressiveness. MT218 in MRMI therefore has the potential for characterizing the invasiveness of solid tumor and assessing tumor response to therapies as well as development of drug resistance. MT218 is also a small, water soluble peptidic monomer with practical industrialization potential and rapid animal pharmacokinetics as required for commercial contrast agents.

In addition to targeted MRI contrast agents, Molecular Theranostics (MT) also developed PET probes and fluorescence probes specific to an oncoprotein in tumor microenvironment. The efficacy of the imaging agents has been demonstrated in various tumor models. The imaging agents are effective to provide early detection of small aggressive solid tumors and able to differentiate aggressive tumors from benign tumors and tissues. Clinical application of these imaging agents has great promise to provide much needed assistance to the physicians for precision cancer management. However, additional methods are needed to diagnose and treat cancer and EMT.

### SUMMARY

The inventors have identified a large number of new targeting peptides that can be used to bind molecular probes to extradomain fibronectins. The present invention provides molecular probe compounds according to the formula P-L-I. P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide. Methods of detecting cancer and monitoring the treatment of cancer using the molecular probes are also provided.

### BRIEF DESCRIPTION OF THE FIGURES

The accompanying drawings, which are incorporated in and constitute a part of this specification, illustrate some embodiments of the inventions, and together with the description, serve to explain principles of the inventions.

FIG. 1 provides a scheme showing the synthesis of ZD2-S0456. The ZD2 peptide was synthesized using standard solid phase peptide synthesis on chlorotrityl resin. S0456 was then conjugated to the N-terminus of ZD2 by adding NHS-S0456 to the ZD2 peptide on resin in the presence of DIPEA in DMF. ZD2-S0456 was then obtained by treating the resin with a cocktail of TFA: H₂O: TIBS for cleavage from the resin and deprotection of the side chain groups. ZD2-S0456 was purified by preparative HPLC and characterized by ESI mass spectrometry (calculated m/z=1748.6, obs. m/z=1748) with a purity exceeding 98% according to analytic HPLC.

FIGS. 2A-2C provide graphs showing the (A) electrospray mass spectrum, (B) HPLC chromatograph and (C) UV spectrum of ZD2-S0456.

FIGS. 3A and 3B provide images showing (A) fluorescence images of the organ tissues of the mice received IV injection of ZD2-S0456, showing ZD2-S0456 binding to HSC3 HNC xenograft. 1: kidney; 2: heart; 3: spleen; 4: brain; 5: muscle; 6: tumor; 7: liver; 8: lung. Fluorescence seen in the kidney, liver, and brain are shown to exhibit autofluorescence rather than binding of the dye. (B) Fluorescence image of mice bearing HSC3 HNC xenografts topically stained with PBS or ZD2-Cy5.5.

FIG. 4 provide a fluorescence image of mouse bearing human breast cancer MDA-MB-231 TNBC xenograft after IV injection of ZD2-S0456, showing ZD2-S0456 staining to the tumor.

FIG. 5 provides representative peptide sequences including deletion or truncation.

FIG. 6 provides representative peptide sequences including C-terminal modification.

FIG. 7 provides representative molecular probes including contrast agents.

FIGS. 8A-8H provides various synthetic routes that can be used to prepare molecular probes including contrast agents.

FIG. 9 provides a synthetic procedure for the near infrared imaging agent ZD2-PPIX.

FIG. 10 provides a synthetic procedure for the fluorescent imaging agent ZD2-S0456.

FIG. 11 provides the structure of the fluorescent imaging agent AVRTSAD-S0456.

FIG. 12 provides a synthetic procedure for the PET imaging agent AVRTSAD-18F.

FIG. 13 provides a synthetic procedure for the PET imaging agent AVRTSAD-HBEDCC-68Ga.

FIG. 14 shows a reaction pathway for compounds C1, C2, and C3.

FIG. 15 shows a reaction pathway for the synthesis of tris(2-nitrobenzenesulfonyl) diethylenetriamine.

FIG. 16 shows a reaction pathway for the synthesis of compound C4.

FIG. 17 shows a reaction pathway for the synthesis of compound 3,6,9,15-(12-propargyloxy)tetraazabicyclo [9.3.1]pentadeca-1 (15), 11,13-triene or PO-pyclen, C5.

FIG. 18 shows a reaction pathway for the synthesis of compound 3,6,9,15-(12-propargyloxy)tetraazabicyclo [9.3.1]pentadeca-1 (15), 11,13-triene-3,6,9,-tri (2′-hydroxymethylacetic acid) or PC3HMA, C6.

FIG. 19 shows the preparation of synthesis of ZD2-PC3HMA.

FIG. 20 shows the addition of manganese to ZD2-(Mn-PC3HMA).

FIG. 21 provides a graph showing the relaxivity for ZD2-(Mn-PC3HMA).

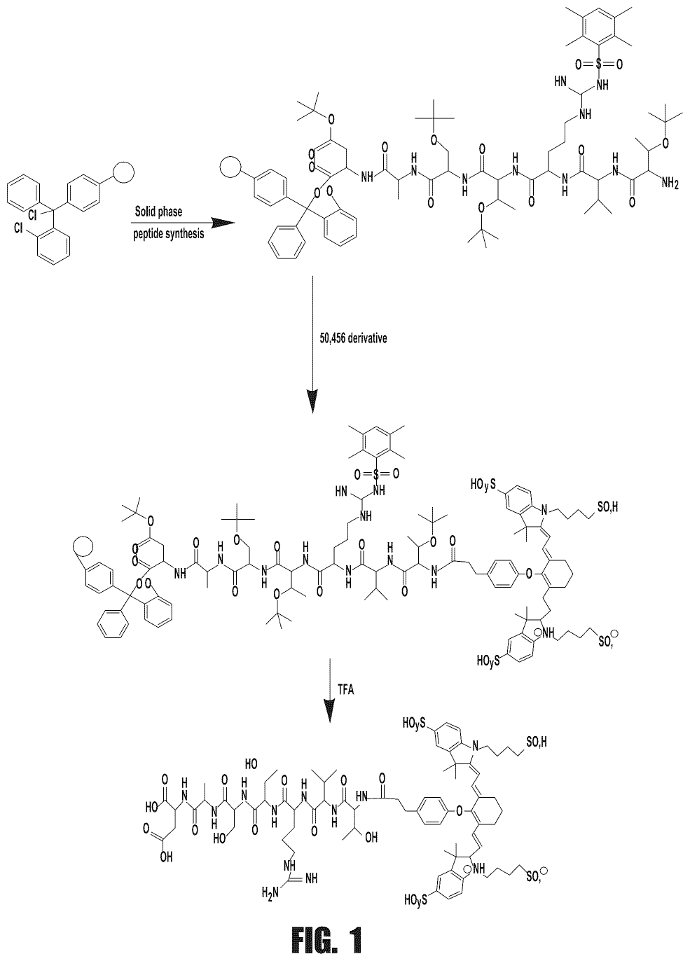

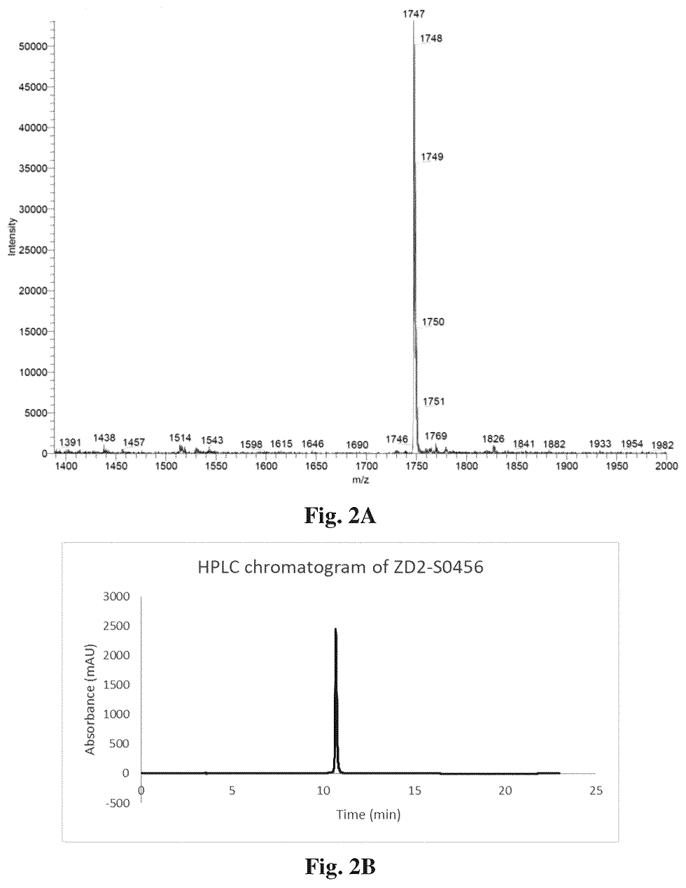

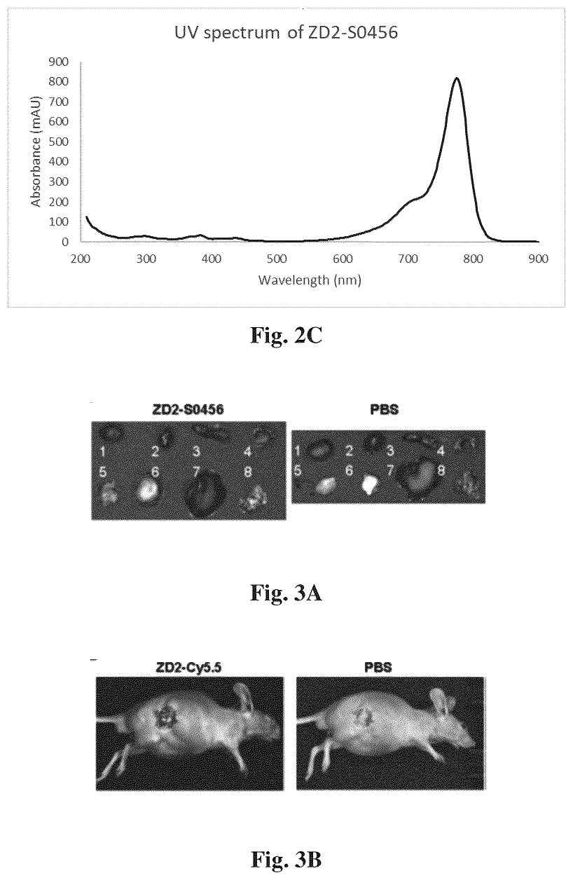

### DETAILED DESCRIPTION

Unless otherwise defined, all technical and scientific terms used herein have the same meaning as commonly understood by one of ordinary skill in the art to which these inventions belong. The terminology used in the description of the inventions herein is for describing particular embodiments only and is not intended to be limiting of the inventions. As used in the description of the inventions and the appended claims, the singular forms “a,” “an,” and “the” are intended to include the plural forms as well, unless the context clearly indicates otherwise.

The numerical ranges and parameters setting forth the broad scope of the inventions are in some cases approximations. Nonetheless, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The term “agent” is used herein to denote a chemical compound, a mixture of chemical compounds, a biological macromolecule, or an extract made from biological materials.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on.

The terms “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Diagnosis of humans is of particular interest.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-cancer activity of the compounds. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

When a group is present more than once in any formula or scheme described herein, each group (or substituent) is independently selected, whether explicitly stated or not. For example, for the formula —C(O)—NR₂ each R group is independently selected.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

“Amino acid” is used herein to refer to a chemical compound with the general formula: NH₂—CRH—COOH, where R, the side chain, is H or an organic group. Where R is organic, R can vary and is either polar or nonpolar (i.e., hydrophobic). The following abbreviations are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine. Unless otherwise indicated, the term “amino acid” as used herein also includes amino acid derivatives that nonetheless retain the general formula.

The term “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

### Molecular Probes

In one aspect, the present invention provides molecular probe compounds according to the following formula:

P-L-I

wherein P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof.

The imaging agent is directly conjugated to the targeting peptide via a linker. The role of the imaging agent is to facilitate the detection step of a detection or diagnostic method by allowing visualization of the complex formed by binding of a molecular probe comprising a targeting peptide to EDB-FN. The imaging agent can be selected such that it generates a signal, which can be measured and whose intensity is related (preferably proportional) to the amount of the molecular probe bound to the tissue being analyzed.

### Targeting Peptides

Molecular probes including the targeting peptides can be administered systemically to a subject, such as by intravenous or parenteral administration, and readily target extradomain fibronectin proteins using the targeting peptides to define cancer cell location, distribution, and/or aggressiveness as well as tumor cell margins in the subject. For a description of similar molecular probes, and how they can be prepared, see U.S. Pat. No. 10,925,980, the disclosure of which is incorporated herein by reference in its entirety.

The targeting peptides described herein include a peptide sequence that specifically binds to and/or complexes with oncofetal fibronectin (onfFN) isoforms, extradomain-B fibronectin (EDB-FN) or extradomain-A (EDA-FN) fibronectin, which can collectively be referred to as extradomain fibronectins. Cancer, and particularly, malignant cancer has a unique tumor microenvironment that facilitates cancer cell survival, proliferation, and metastasis. The presence of onfFN has been shown in various human cancer types, including prostate and breast cancer.

High expression of onfFN, EDB-FN and/or EDA-FN, inversely correlated with cancer aggressiveness and patient survival. It was found that molecular probes that include target peptides which specifically bind to EDB-FN and/or EDB-FN can be used for detecting, monitoring, imaging, and/or treating cancer in the tissue of a subject as well as to determine cancer cell aggressiveness, malignancy, metastasis, migration, dispersal, and/or invasion.

The targeting peptide provides the ability of the molecular probe to specifically bind to an extradomain fibronectin. The targeting peptides can include from 6 to 10 amino acids. Targeting peptides that specifically bind to extradomain fibronectins can include linear peptides having the amino acid sequences of TVRTSAD (SEQ ID NO: 1), VTRTSAD (SEQ ID NO: 2), AVRTSAD (SEQ ID NO: 3), TARTSAD (SEQ ID NO: 4), TVATSAD (SEQ ID NO: 5), TVRASAD (SEQ ID NO: 6), TVRTSAA (SEQ ID NO: 7), HVRTSAD (SEQ ID NO: 8), THRTSAD (SEQ ID NO: 9), TVHTSAD (SEQ ID NO: 10), TVRHSAD (SEQ ID NO: 11), TVRTSAH (SEQ ID NO: 12), HVRTSAD (SEQ ID NO: 13), AHRTSAD (SEQ ID NO: 14), AVHTSAD (SEQ ID NO: 15), AVRHSAD (SEQ ID NO: 16), AVRTHAD (SEQ ID NO: 17), AVRTSAH (SEQ ID NO: 18), AARTSAD (SEQ ID NO: 19), AVATSAD (SEQ ID NO: 20), AVRASAD (SEQ ID NO: 21), AVRTAAD (SEQ ID NO: 22), AVRTSAA (SEQ ID NO: 23), NVRTSAD (SEQ ID NO: 24), TNRTSAD (SEQ ID NO: 25), TVNTSAD (SEQ ID NO: 26), TVRNSAD (SEQ ID NO: 27), TVRTSAN (SEQ ID NO: 28), NVRTSAD (SEQ ID NO: 29), ANRTSAD (SEQ ID NO: 30), AVNTSAD (SEQ ID NO: 31), AVRNSAD (SEQ ID NO: 32), AVRTNAD (SEQ ID NO: 33), AVRTSAN (SEQ ID NO: 34), TV*RTSAD (* indicates a D-amino acid) (SEQ ID NO: 35), TVR*TSAD (SEQ ID NO: 36), TVRT*SAD (SEQ ID NO: 37), TVRTS*AD (SEQ ID NO: 38), TVRTSA*D (SEQ ID NO: 39), TVRTSAD* (SEQ ID NO: 40), AV*RTSAD (SEQ ID NO: 41), AVR*TSAD (SEQ ID NO: 42), AVRTSAD* (SEQ ID NO: 43), AVR*TSAH (SEQ ID NO: 44), TVRTSA (SEQ ID NO: 45), AVRTSA (SEQ ID NO: 46), VRTSAD (SEQ ID NO: 47), NWGDRIL (SEQ ID NO: 48), AWGDRIL (SEQ ID NO: 49), NAGDRIL (SEQ ID NO: 50), NWADRIL (SEQ ID NO: 51), NWGARIL (SEQ ID NO: 52), NWGDAIL (SEQ ID NO: 53), NWGDRAL (SEQ ID NO: 54), NWGDRIA (SEQ ID NO: 55), NWGKPIK (SEQ ID NO: 56), AWGKPIK (SEQ ID NO: 57), NAGKPIK (SEQ ID NO: 58), NWAKPIK (SEQ ID NO: 59), NWGAPIK (SEQ ID NO: 60), NWGKAIK (SEQ ID NO: 61), NWGKPAK (SEQ ID NO: 62), NWGKPIA (SEQ ID NO: 63), SGVKSAF (SEQ ID NO: 64), AGVKSAF (SEQ ID NO: 65), SAVKSAF (SEQ ID NO: 66), SGAKSAF (SEQ ID NO: 67), SGVASAF (SEQ ID NO: 68), SGVKAAF (SEQ ID NO: 69), SGVKSAA (SEQ ID NO: 70), GVKSYNE (SEQ ID NO: 71), AVKSYNE (SEQ ID NO: 72), GAKSYNE (SEQ ID NO: 73), GVASYNE (SEQ ID NO: 74), GVKAYNE (SEQ ID NO: 75), GVKSANE (SEQ ID NO: 76), GVKSYAE (SEQ ID NO: 77), GVKSYNA (SEQ ID NO: 78), IGKTNTL (SEQ ID NO: 79), AGKTNTL (SEQ ID NO: 80), JAKTNTL (SEQ ID NO: 81), IGATNTL (SEQ ID NO: 82), IGKANTL (SEQ ID NO: 83), IGKTATL (SEQ ID NO: 84), IGKTNAL (SEQ ID NO: 85), IGKTNAA (SEQ ID NO: 86), IGNSNTL (SEQ ID NO: 87), AGNSNTL (SEQ ID NO: 88), IANSNTL (SEQ ID NO: 89), IGASNTL (SEQ ID NO: 90), IGNANTL (SEQ ID NO: 91), IGNSATL (SEQ ID NO: 92), IGNSNAL (SEQ ID NO: 93), IGNSNTA (SEQ ID NO: 94), IGNTIPV (SEQ ID NO: 95), AGNTIPV (SEQ ID NO: 96), IANTIPV (SEQ ID NO: 97), IGATIPV (SEQ ID NO: 98), IGNAIPV (SEQ ID NO: 99), IGNTAPV (SEQ ID NO: 100), IGNTIAV (SEQ ID NO: 101), IGNTIPA (SEQ ID NO: 102), LYANSPF (SEQ ID NO: 103), AYANSPF (SEQ ID NO: 104), LAANSPF (SEQ ID NO: 105), LYAASPF (SEQ ID NO: 106), LYANAPF (SEQ ID NO: 107), LYANSAF (SEQ ID NO: 108), LYANSPA (SEQ ID NO: 109), AVR*TSAN (SEQ ID NO: 110), ARTSAD (SEQ ID NO: 111), TLRTSAD (SEQ ID NO: 112), ALRTSAH (SEQ ID NO: 113), TLRTSAT (SEQ ID NO: 114), ALRTSAT (SEQ ID NO: 115), TLRTSAS (SEQ ID NO: 116), ALRTSAS (SEQ ID NO: 117). In some embodiments, wherein the targeting peptide comprises SEQ ID NO: 1 or SEQ ID NO: 2.

The targeting peptides can be subject to various changes, substitutions, insertions, and deletions where such changes provide for certain advantages in its use. In this regard, targeting peptides that bind to and/or complex with EDB-FN can be substantially homologous with, rather than be identical to, the sequence of a recited peptide where one or more changes are made and it retains the ability to function as specifically binding to and/or complexing with extradomain fibronectin.

In further embodiments, the targeting peptide can be coupled to a Fc region of an IgG to form a targeting peptide-Fc chimera that can specifically bind to EDB-FN and/or EDA-FN. Advantageously, the targeting peptide-Fc chimera can induce immune responses, such as complement-dependent lysis and antibody-dependent cellular cytotoxicity, that target tumor cells thereby eliciting anti-tumor activities.

Chimeric proteins that can combine the Fc regions of IgG with one or more domains of another protein, such as various cytokines and soluble receptors, are known. These chimeric proteins can be fusions of human Fc regions and human domains of another protein. These chimeric proteins would then be a “humanized Fc chimera”. See, for example, Capon et al., Nature, 337:525-531, 1989; Chamow et al., Trends Biotechnol., 14:52-60, 1996); U.S. Pat. Nos. 5,116,964 and 5,541,087. The chimeric protein can be a homodimeric protein linked through cysteine residues in the hinge region of IgG Fc, resulting in a molecule similar to an IgG molecule without the CHI domains and light chains. Due to the structural homology, such Fc fusion proteins exhibit in vivo pharmacokinetic profile comparable to that of human IgG with a similar isotype.

The term “analog” includes any peptide having an amino acid residue sequence substantially identical to a sequence specifically shown herein in which one or more residues have been conservatively substituted with a functionally similar residue and that specifically binds to and/or complexes with EDA-FN or EDB-FN as described herein. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Peptides described herein also include any peptide having one or more additions and/or deletions or residues relative to the sequence of a peptide whose sequence is shown herein, so long as the requisite binding specificity or activity is maintained.

The targeting peptides can be synthesized by any of the techniques that are known to those skilled in the polypeptide art, including recombinant DNA techniques. Synthetic chemistry techniques, such as a solid-phase Merrifield-type synthesis, can be used for reasons of purity, antigenic specificity, freedom from undesired side products, case of production and the like. A summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as an example, the protected or derivatized amino acid can be attached to an inert solid support through its unprotected carboxyl or amino group. The protecting group of the amino or carboxyl group can then be selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group can then be removed from this newly added amino acid residue, and the next amino acid (suitably protected) is then added, and so forth. After all the desired amino acids have been linked in the proper sequence, any remaining terminal and side group protecting groups (and solid support) can be removed sequentially or concurrently, to afford the final linear polypeptide.

### Imaging Agents

The molecular probes include an imaging agent (I). The role of the imaging agent is to facilitate the detection step of a detection or diagnostic method by allowing visualization of the complex formed by binding of a molecular probe comprising a targeting peptide to EDB-FN and/or EDA-FN. The imaging agent includes a detectable moiety can be selected such that it generates a signal, which can be measured and whose intensity is related (preferably proportional) to the amount of the molecular probe bound to the tissue being analyzed. The imaging agent is typically connected to the targeting peptide through a linking molecule.

Imaging agents, as used herein, include chemical agents that can be used to image cancer in a subject, but can also include chemical agents that can be used to treat cancer in a subject, such as radioactive compounds. In some embodiments, I is a magnetic resonance imaging (MRI), positron emission tomography (PET), or single photon emission computed tomography (SPECT) contrast agent. In some embodiment, the I is a dye that has a fluorescence excitation and emission spectra in the near infra-red (NIR) range, and the compound maintains or enhances the fluorescence of the dye. NIR dyes can be selected from the group consisting of LS288, IR800, SP054, S0121, KODAK IRD28, S2076, S0456 and derivatives thereof. In some embodiments, I is a radioactive imaging or therapeutic compound.

Any of a wide variety of detectable moieties can be used with the targeting peptides described herein. Examples of detectable moieties include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

In some embodiments, the molecular probe includes an imaging group suitable for use as a magnetic resonance imaging agent. Disease detection using MRI is often difficult because areas of disease have similar signal intensity compared to surrounding healthy tissue. In the case of magnetic resonance imaging, the imaging agent can also be referred to as a contrast agent. Lanthanide elements are known to be useful as contrast agents. The lanthanide chemical elements comprise the fifteen metallic chemical elements with atomic numbers 57 through 71, and include lanthanum, cerium, prascodymium, neodymium, promethium, samarium, europium, gadolinium, terbium, dysprosium, holmium, erbium, thulium, ytterbium, and lutetium. Preferred lanthanides include europium, gadolinium, and terbium. In order to more readily handle these rare earth metals, the lanthanides are preferably chelated. In some embodiments, the lanthanide selected for use as an imaging group is gadolinium, or more specifically gadolinium (III).

The selection of the metal ion can vary depending upon the detection technique (e.g., MRI, PET, etc.). In one aspect, metal ions useful in magnetic resonance imaging include Gd⁺³, Eu⁺³, Tm⁺³, Dy⁺³, Yb⁺³, Mn⁺², or Fe⁺³ ions. In another aspect, ions useful in PET and SPECT imaging include ⁵⁵Co, ⁶⁴Cu, ⁶⁷Cu, ⁴⁷Sc, ⁶⁶Ga, ⁶⁸Ga, ⁹⁰Y, ⁹⁷Ru, ^{99m}Tc, ¹¹¹In, ¹⁰⁹Pd, ¹⁵³Sm, ¹⁷⁷Lu, ¹⁸⁶Re, ¹⁸⁸Rc. In another aspect, the imaging agent comprises an MRI agent, wherein the MRI agent comprises a chelating agent and a metal ion comprising Gd⁺³, Eu⁺³, Tm⁺³, Dy⁺³, Yb⁺³, Mn⁺², or Fe⁺³ ions.

In some embodiments, the imaging agent is a PET imaging agent. In one example, the detectable moiety can include a radiolabel, that is coupled (e.g., attached or complexed) with the targeting peptides using general organic chemistry techniques. Radiolabels, such as ¹²³I, ¹³¹I, ¹²⁵I, ¹⁸F, ¹¹C, ⁷⁵Br, ⁷⁶Br, ¹²⁴I, ¹³N, ⁶⁴Cu, ³²P, ³⁵S, can be used for PET techniques imaging by well-known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, N Y 1986) the contents of which are hereby incorporated by reference. The detectable moiety can also include ¹²³I for SPECT. The ¹²³I can be coupled to the targeting peptide by any of several techniques known to the art. See, e.g., Kulkarni, Int. J. Rad. Appl. & Inst. (Part B) 18:647 (1991), the contents of which are hereby incorporated by reference. In addition, the detectable moiety can include any radioactive iodine isotope, such as, but not limited to ¹³¹I, ¹²⁵I, or ¹²³I. The radioactive iodine isotopes can be coupled to the targeting peptide by iodination of a diazotized amino derivative directly via a diazonium iodide, or by conversion of the unstable diazotized amine to the stable triazene, or by conversion of a non-radioactive halogenated precursor to a stable tri-alkyl tin derivative, which then can be converted to the iodo compound by several methods well known to the art.

For the purposes of in vivo imaging, the type of detection instrument available is a major factor in selecting a given detectable moiety. For instance, the type of instrument used will guide the selection of a stable isotope. The half-life should be long enough so that it is still detectable at the time of maximum uptake by the target, but short enough so that the host does not sustain deleterious effects.

The detectable moiety can further include known metal radiolabels, such as Technetium-99m (^{99m}Tc), ¹⁵³Gd, ¹¹¹In, ⁶⁷Ga, ²⁰¹Tl, ⁶⁸Ga, ⁸²Rb, ⁶⁴Cu, ⁹⁰Y, ¹⁸⁸Rh, T (tritium), ¹⁵³Sm, ⁸⁹Sr, ¹⁷⁷Lu, and ²¹¹At. Modification of the targeting peptides to introduce ligands that bind such metal ions can be affected without undue experimentation by one of ordinary skill in the radiolabeling art. The metal radiolabeled molecular probes can then be used to detect cancers, such as prostate cancer in the subject. Preparing radiolabeled derivatives of Tc99m is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26 (2): 217-24, (1999); Oya et al., “Small and neutral Tc (v) O BAT, bisaminocthanethiol (N2S2) complexes for developing new brain imaging agents” Nuclear Medicine & Biology 25 (2): 135-40, (1998); and Hom et al., “Technetium-99m-labeled receptor-specific small-molecule radiopharmaceuticals: recent developments and encouraging results” Nuclear Medicine & Biology 24 (6): 485-98, (1997).

In some embodiments, the imaging agent is a visual agent such as a fluorescent or near infrared imaging agent. Fluorescent labeling agents or infrared agents include those known in the art, many of which are commonly commercially available, for example, fluorophores, such as ALEXA 350, PACIFIC BLUE, MARINA BLUE, ACRIDINE, EDANS, COUMARIN, BODIPY 493/503, CY2, BODIPY FL-X, DANSYL, ALEXA 488, FAM, OREGON GREEN, RHODAMINE GREEN-X, TET, ALEXA 430, CAL GOLD™, BODIPY R6G-X, JOE, ALEXA 532, VIC, HEX, CAL ORANGE™, ALEXA 555, BODIPY 564/570, BODIPY TMR-X, QUASAR™ 570, ALEXA 546, TAMRA, RHODAMINE RED-X, BODIPY 581/591, CY3.5, ROX, ALEXA 568, CAL RED, BODIPY TR-X, ALEXA 594, BODIPY 630/650-X, PULSAR 650, BODIPY 630/665-X, ALEXA 647, IR800, and QUASAR 670. Fluorescent labeling agents can include other known fluorophores, or proteins known in the art, for example, green fluorescent protein. The disclosed targeting peptides can be coupled to the fluorescent labeling agents, administered to a subject or a sample, and the subject/sample examined by fluorescence spectroscopy or imaging to detect the labeled compound.

In some embodiments, the imaging agent includes a chelating agent and a metal ion. The chelating agent generally possesses one or more groups capable of forming a covalent bond with the peptide. A number of different chelating agents known in the art can be used herein. In one aspect, the chelating agent comprises an acyclic or cyclic compound comprising at least one heteroatom (e.g., oxygen, nitrogen, sulfur, phosphorous) that has lone-pair electrons capable of coordinating with the imaging agent. An example of an acyclic chelating agent includes ethylenediamine. Examples of cyclic chelating agents include diethylenetriaminepentaacetate (DTPA) or its derivatives, 1,4,7,10-tetraazadodecanetetraacetate (DOTA) and its derivatives, 1,4,7,10-tetraazadodecane-1,4,7-triacetate and (DO3A) its derivatives, derivatives, 1,4,7,10-tetraazacyclotridecanetetraacetic acid (TRITA) and its derivatives, 1,4,8,11-tetraazacyclotetradecane-1,4,8,11-tetraacetic acid (TETA) and its derivatives, 1,4,7,10-tetraazadodecanetetramethylacetate (DOTMA) and its derivatives, 1,4,7,10-tetraazadodecane-1,4,7-trimethylacetate (DO3MA) and its derivatives, N,N′,N″,N″-tetraphosphonatomethyl-1,4,7,10-tetraazacyclododecane (DOTP) and its derivatives, 1,4,7,10-tetraazacyclododecane-1,4,7,10-tetrakis(methylene methylphosphonic acid) (DOTMP) and its derivatives, 1,4,7,10-tetraazacyclododecane-1,4,7,10-tetrakis(methylene phenylphosphonic acid) (DOTPP) and its derivatives, or N,N′-ethylenedi-L-cysteine or its derivatives. The term “derivative” is defined herein as the corresponding salt and ester thereof of the chelating agent.

In some embodiments, the imaging compound is according to formula I:

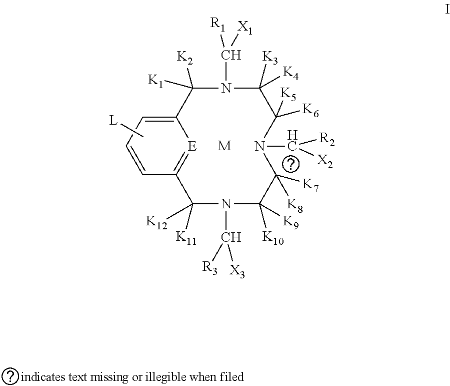

wherein: R₁, R₂ and R₃ represent, independently of one another, —COOH, —P(O)(OH)₂ or —R₆-P(O)—OH in which R₆ represents an H atom or a C₁-C₃ alkyl group, preferably COOH; X₁, X₂ and X₃ represent, independently of one another, —(CH₂) OH, with a,b=1 to 3, advantageously —CH₂OH, —CH₂—CH₂OH, —CHOH—CH₂OH, —CH—(CH₂OH)₂, or L-Y in which L represents a C1-C3 alkyl group, preferably (CH₂) n with n=1 to 3, Y represents CO—NR₇R₈, in which R₇ represents H or a C1-C6 alkyl group or a C1-C6 hydroxyalkyl group, in particular a C2-C4 group, advantageously —CH₂—CH₂OH, —CHOH—CH₂OH, —CH—(CH₂OH)₂, —(CH₂)ₘ—(CHOH)ₚ—CH₂OH, with m=1 to 3, p=1 to 4 and m+p=2 to 5, or —C—(CH₂OH)₃, and R₈ represents a C1-C6 alkyl or C1-C6 hydroxyalkyl group, in particular a C2-C4 group, advantageously —CH₂—CH₂OH, —CHOH—CH₂OH, —CH—(CH₂OH)₂, —(CH₂)ₘ—(CHOH)ₚ—CH₂OH, with m=1 to 3, p=1 to 4 and m+p=2 to 5, or —C—(CH₂OH)₃, provided that at least R₇ or R₈ represents a C1-C6 hydroxyalkyl group; D represents CH or N; E represents CH or N; F₁ represents CH or N; K₁ to K₁₂—each independently represent H, —(CH₂)j-CH₃ or —(CH₂)i-OH, in which j=0 to 3 and i=1 to 3, advantageously H, or K₃ or K₄ with K₅ or K₆, and/or K₇ or K₈ with K₉ or K₁₀, form a ring having 3 to 6 carbon atoms; and M is a paramagnetic ion selected from Gd⁺³, Eu⁺³, Tm⁺³, Dy⁺³, Yb⁺³, Mn⁺², and Fe⁺³.

In some embodiments, K₁-K₁₂ are H. In further embodiments, D is C, E is N, and F1 is CH. In additional embodiments, R₁, R₂, and R₃ are —COOH. In yet further embodiments, X₁, X₂, and X₃ are —CH₂OH. In some embodiments, the paramagnetic ion M is Mn²⁺ or Gd³⁺.

In some embodiments, the imaging agent is a fluorescent imaging agent. An example of a suitable fluorescent imaging agent is an imaging agent according to formula II:

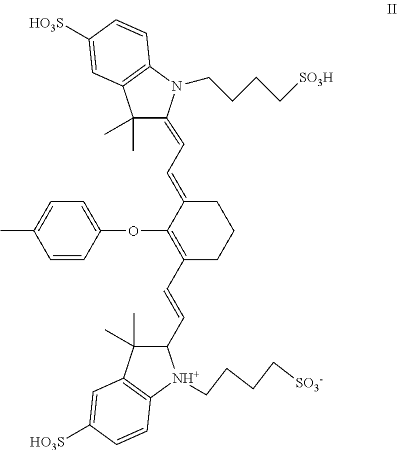

### Linkers

Additional residues may also be added at either terminus of the targeting peptide to provide a “linker” by which the peptides can be conveniently linked and/or affixed to the imaging agent and/or other polypeptides, proteins, labels, solid matrices, or carriers. Conjugation of the targeting peptide through the linker can be through the meta- or para-position of the pyridine ring of the imaging agent.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject targeting peptide agent can differ by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the non-peptide linker is a non-peptide aliphatic or heteroaliphatic linker. The non-peptide linker can include an alkylene dicarboxamide that covalently links the peptide and contrast agent.

In some embodiments, the non-peptide linker can include a first portion that is about 1 to about 10 atoms in lengths and second portion that acts as a spacer. The portion of the linker that acts a spacer can include a non-peptide polymer that includes but is not limited to a polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharides, dextran, polyvinylpyrrolidones, polyvinyl ethyl ether, polyacryl amide, polyacrylate, polycyanoacrylates, lipid polymers, chitins, hyaluronic acid, and heparin. For more detailed descriptions of spacers for non-peptide linkers, see, for example, WO/2006/107124, which is incorporated by reference herein. Typically such linkers will have a range of molecular weight of from about 1 kDa to 50 kDa, depending upon a particular linker. For example, a typical PEG has a molecular weight of about 1 to 5 kDa, and polyethylene glycol has a molecular weight of about 5 kDa to 50 kDa, and more preferably about 10 kDa to 40 kDa.

In some embodiments, the linker comprises at least one of polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharide, dextran, polyvinylpyrrolidone, polyvinyl ethyl ether, polyacryl amide, polyacrylate, or polycyanoacrylate.

### Methods of Detecting Cancer

A further aspect of the present invention provides a method of detecting cancer in a subject. The method includes contacting a tissue of the subject with an effective amount of a molecular probe comprising the formula:

P-L-I

wherein P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof; detecting the amount of the molecular probe present in the tissue; comparing the amount of molecular probe detected to a control value; and detecting cancer in the subject if the amount of the molecular probe present in the tissue is higher than the control value.

Molecular probes can be used in a method to detect and/or determine the presence, location, and/or distribution of cancer cells expressing extradomain fibronectin in an organ, tissue, or body area of a subject. The presence, location, and/or distribution of the molecular probe in the subject's tissue, e.g., prostate tissue, can be visualized (e.g., with an in vivo imaging modality described herein). “Distribution” as used herein is the spatial property of being scattered about over an area or volume. In this case, “the distribution of cancer cells” is the spatial property of cancer cells being scattered about over an area or volume included in the subject's tissue, e.g., prostate tissue. The distribution of the molecular probe may then be correlated with the presence or absence of cancer cells in the tissue.

In some embodiments, the molecular probes described herein may be used in conjunction with non-invasive imaging techniques for in vivo imaging of the molecular probe, such as magnetic resonance spectroscopy (MRS) or imaging (MRI), or gamma imaging, such as positron emission tomography (PET), single-photon emission computed tomography (SPECT), CT contrast image, or ultrasound imaging. The term “in vivo imaging” refers to any method, which permits the detection of a labeled targeting peptide, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of molecular probe along with a large excess of unlabeled, but otherwise chemically identical compound.

In one aspect, the molecular probes may be administered to a subject to assess the distribution of cancer cells in a subject and correlate the distribution to a specific location. Surgeons routinely use stereotactic techniques and intra-operative MRI (iMRI) in surgical resections. This allows them to specifically identify and sample tissue from distinct regions of the tumor such as the tumor edge or tumor center. Frequently, they also sample regions of tissue on the tumor margin that are outside the tumor edge that appear to be grossly normal but are infiltrated by dispersing tumor cells upon histological examination.

The terms “cancer” or “tumor” refer to any neoplastic growth in a subject, including an initial tumor and any metastases. The cancer can be of the liquid or solid tumor type. Types of cancer include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma. In some embodiments, the cancer is breast cancer, oral cancer, pancreatic cancer, or prostate cancer.

In some embodiments, the invention provides methods of detecting and monitoring treatment of drug-resistant cancer, which is often associated with extradomain fibronectin. Drug-resistant cancer is a type of cancer that has developed resistance to treatment with anticancer agents. The various facets of drug resistance are known to those skilled in the art. See Vasan et al., Nature volume 575, 299-309 (2019). Drug-resistant cancer can include intrinsic and acquired resistance. Examples of factors contributing to drug resistance include tumor burden, tumor heterogeneity, the generation or use of physical barriers by the tumor, immunosuppression, and undruggable genomic drivers.

The method includes the step of contacting a tissue of the subject. Contacting, as used herein, refers to causing two items to become physically adjacent and in contact, or placing them in an environment where such contact will occur within a reasonably short timeframe. For example, contacting a tissue with a molecular probe includes directly applying the molecular probe to a tissue, such as a biopsy sample that has been obtained from a subject. Contacting can include contacting in vivo, ex vivo, and in vitro. However, contacting also includes systemic administration which results in contact between the molecular probe and the tissue through circulation-mediated contact. Accordingly, in some embodiments, the tissue is contacted in vivo.

In some embodiments, the molecular probe is systemically administered to a subject having or suspected of having cancer. General signs and symptoms associated with cancer include fatigue, weight changes, or a lump or area of thickening that can be felt under the skin. Most cancer signs and symptoms are specific to the tissue in which cancer has occurred. For example, headaches or seizures can be a sign of brain cancer, while trouble urinating can be a sign of bladder cancer. Nonetheless, the various signs and symptoms of cancer are well-known to those skilled in the art, and are described on the National Cancer Institute Website. Symptoms of cancer can indicate that a subject has or is suspected of having cancer, while other risk factors such as genetic predisposition and exposure to radiation can also lead a subject to being suspected of having cancer.

A tissue region being evaluated using the molecular probes is an area of tissue in the subject which is being treated and/or analyzed. Generally, the tissue region is within the tissue where cancer has been identified, or tissues where it is suspected that cancer may have spread through metastasis. The tissue region can be an organ of a subject such as the heart, lungs, or blood vessels. In other embodiments, the tissue region can be diseased tissue, or tissue that is suspected of being diseased, such as a tumor or tissue regions connected with the tumor by a metastatic route or a tissue having similar characteristics to the primary tumor tissue. Examples of metastatic routes include the transcoelomic route (penetration of the surface of the peritoneal, pleural, pericardial, or subarachnoid space), lymphatic route (transport of tumor cells to lymph nodes and from there to other parts of the body), and the haematogenous route (used by sarcomas and carcinomas). The tissue region can vary widely in size, and can for example range from a size of about 1 cm³ to about 500 cm³.

Molecular probes that specifically bind to and/or complex with extradomain fibronectin associated with malignant or metastatic cells can be used in intra-operative imaging techniques to guide surgical resection and eliminate the “educated guess” of the location of the tumor margin by the surgeon. Previous studies have determined that more extensive surgical resection improves patient survival. Thus, molecular probes that function as diagnostic molecular imaging agents have the potential to increase patient survival rates.

Another embodiment described herein relates to a method of determining the aggressiveness or malignancy of cancer cells in a subject. It was found that the binding intensity of the molecular probes to a cancer correlated with the cancer aggressiveness. Enhanced binding correlated with more aggressive cancer whereas lower or reduced binding correlated with less aggressive or benign tumors. In one example, binding of the molecular probe to prostate tumor sections correlated with to Gleason score based on tumor aggressiveness, where enhanced binding intensity of the molecular probe correlated to aggressive or malignant prostate cancer and which was distinguished from benign prostatic hyperplasia, which displayed lower binding intensity of the probe. The methods and molecular probes described herein can be used to monitor and/or compare the aggressiveness a cancer in a subject prior to administration of a cancer therapeutic or cancer therapy, during administration, or post therapeutic regimen.

In some embodiments, the molecular probe bound to and/or complexed with the EDB-FN and/or EDA-FN is detected in the subject to detect and/or provide the aggressiveness, location and/or distribution of the cancer cells in the subject. The aggressiveness, location and/or distribution of the cancer cells in the subject can then be compared to a control to determine the efficacy of the cancer therapeutic and/or cancer therapy. The control can be the location and/or distribution of the cancer cells in the subject prior to the administration of the cancer therapeutic and/or cancer therapy. The location and/or distribution of the cancer cells in the subject prior to the administration of the cancer therapeutic and/or cancer therapy can be determined by administering the molecular probe to the subject and detecting the molecular probe bound to and/or complexed with cancer cells in the subject prior to administration of the cancer therapeutic and/or cancer therapy.

In some embodiments, to identify and facilitate removal of cancers cells, microscopic intra-operative imaging (IOI) techniques can be combined with systemically administered or locally administered molecular probes described herein. The molecular probe upon administration to the subject can target and detect and/or determine the presence, location, and/or distribution of drug-resistant cancer cells, i.e., cancer cells associated with EDB-FN expression, in an organ or body area of a patient. In one example, the molecular probe can be combined with IOI to identify malignant cells that have infiltrated and/or are beginning to infiltrate at a tumor margin. The method can be performed in real-time during surgery. The method can include local or systemic application of the molecular probe that includes a detectable moiety, such as a PET, fluorescent, or MRI contrast moiety. An imaging modality can then be used to detect and subsequently gather image data. The resultant image data may be used to determine, at least in part, a surgical and/or radiological treatment. Alternatively, this image data may be used to control, at least in part, an automated surgical device (e.g., laser, scalpel, micromachine) or to aid in manual guidance of surgery. Further, the image data may be used to plan and/or control the delivery of a therapeutic agent (e.g., by a micro-electronic machine or micro-machine).

The molecular probe used in the method can be any of the molecular probes described herein. In some embodiments, the molecular probe comprises a contrast agent. In further embodiments, the contrast agent includes a paramagnetic ion selected from the group consisting of Gd⁺³, Eu⁺³, Tm⁺³, Dy⁺³, Yb⁺³, Mn⁺², and Fe⁺³; and salts thereof. In some embodiments, the molecular probe comprises a near infrared imaging agent. In other embodiments, the molecular probe comprises a fluorescent imaging agent. In yet further embodiments, the molecular probe comprises a PET imaging agent.

### Methods of Monitoring Treatment of Drug-Resistant Cancer

In another aspect of the present invention, the methods and molecular probes described herein can be used to monitor the treatment of a subject having cancer. In this embodiment, the tissue of the subject is contacted with an effective amount of a molecular probe a first time prior to, during, or post administration of the therapeutic regimen and the amount and/or distribution of cancer cells can be imaged to determine the efficacy of the treatment. Then, at a later time, the tissue of the subject is contacted with an effective amount of the molecular probe for a second time, and a second amount and/or distribution of the molecular probe present in the tissue is detected. The first amount and the second amount of the molecular probe (and/or its distribution) are then compared to monitor the treatment of the drug resistant cancer in the subject.

More specifically, the present invention provides a method of monitoring the treatment of cancer in a subject. The method includes contacting a tissue of a subject undergoing treatment of cancer with an effective amount of a molecular probe comprising the formula:

P-L-I

for a first time, wherein: P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof; detecting a first amount of the molecular probe present in the tissue; contacting the tissue of the subject for a second time with an effective amount of the molecular probe comprising the formula P-L-I; detecting a second amount of the molecular probe present in the tissue; and comparing the first amount and the second amount of the molecular probe to monitor the treatment of cancer in the subject. In some embodiments, the tissue is contacted in vivo, while in other embodiments the tissue is contacted in vitro.

In some embodiments, the molecular probes described herein can be used to measure the efficacy of a therapeutic administered to a subject for treating cancer, including metastatic or aggressive cancer. In this embodiment, the molecular probe can be administered to the subject prior to, during, or post administration of the therapeutic regimen and the distribution of cancer cells can be imaged to determine the efficacy of the therapeutic regimen. In one example, the therapeutic regimen can include a surgical resection of the cancer and the molecular probe can be used to define the distribution of the cancer pre-operative and post-operative to determine the efficacy of the surgical resection. Optionally, the methods and molecular probes can be used in an intra-operative surgical procedure, such as a surgical tumor resection, to more readily define and/or image the cancer cell mass or volume during the surgery.

The type of cancer being treated can be any of the types of cancer described herein. In some embodiments, the cancer is drug-resistant cancer. In further embodiments, the cancer is breast cancer, oral cancer, pancreatic cancer, or prostate cancer.

The treatment being monitored can be administration of a cancer therapeutic or the use of other types of cancer therapy. A “cancer therapeutic,” as used herein, can include any agent that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapy can include such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy. This can provide a direct clinical efficacy endpoint measure of a cancer therapeutic. Therefore, in another aspect, a method of monitoring the efficacy of a cancer therapeutic is provided. More specifically, embodiments of the application provide for a method of monitoring the efficacy of a cancer therapy.

The therapeutic agent can include an anti-proliferative agent that exerts an antincoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in preclinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

Special methods can be used to treat drug-resistant cancer. One method of treating drug-resistant cancer is to use combination therapy in which a plurality of chemotherapeutic agents having non-overlapping mechanisms of action are used. See Bosl et al., N. Eng. J. Med., 294, 405-410 (1986). Drug-resistant cancer can also be treated by varying dose intensity or using high doses of chemotherapy. See Sternberg et al., J. Clin. Oncol., 19, 2638-2646 (2001). Other methods include the use of therapies specifically targeted to enabling characteristics of the drug-resistant cancer, such as targeting tyrosine kinase, nuclear receptors, or estrogen receptors. See Hanahan D., Weinberg R. A., Cell, 144, 646-674 (2011). Immunological approaches, and in particular the use of monoclonal antibodies to disable immune checkpoints, can also be used to treat drug resistant cancer. See Ribas A., Wolchok J. D., Science, 359, 1350-1355 (2018).

The molecular probe used to monitor or evaluate treatment can be any of the molecular probes described herein. In some embodiments, the molecular probe comprises a contrast agent. In further embodiments, the contrast agent includes a paramagnetic ion selected from the group consisting of Gd⁺³, Eu⁺³, Tm⁺³, Dy⁺³, Yb⁺³, Mn⁺², Fe⁺³; and salts thereof. In some embodiments, the molecular probe comprises a near infrared imaging agent. In further embodiments, the molecular probe comprises a fluorescent imaging agent. In yet further embodiments, the molecular probe comprises a PET imaging agent.

### Methods of Cancer Treatment

In another aspect, the present invention provides a method of treating cancer in a subject, in which the targeting peptides are conjugated to a therapeutic agent and administered to a subject for treating a cancer, such as a metastatic cancer. More specifically, a method of treating cancer in a subject is provided that includes administering a therapeutically effective amount of a molecular probe comprising the formula:

P-L-T

to the subject, wherein P is a targeting peptide selected from the group consisting of SEQ ID NO: 1 to SEQ ID NO: 117; T is a therapeutic agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof.

In some embodiments, the therapeutic agent is a radioactive compound held by a chelating agent, in a manner similar to that described for contrast agents. In other embodiments, the therapeutic agent is any anti-proliferative agent that can be readily bound to the linker peptide. A wide variety of anti-proliferative agents are known to those skilled in the art, and described herein.

### Administration and Formulation

The molecular probe described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the molecular probe can be by intravenous injection of the molecular probe in the subject. Single or multiple administrations of the probe can be given. “Administered”, as used herein, means provision or delivery of a molecular probe in an amount(s) and for a period of time(s) effective to label cancer cells in the subject.

Molecular probes comprising the targeting peptides described herein can be administered to a subject in an effective amount of a pharmaceutical composition containing a molecular probe or a pharmaceutically acceptable water-soluble salt thereof, to a patient.

An “effective amount” means that the amount of the molecular probe that is administered is sufficient to enable detection of binding or complexing of the probe to EDB-FN and/or EDA-FN expressed by the cancer cells or other cells in the cancer cell microenvironment. An “imaging effective quantity” means that the amount of the molecular probe that is administered is sufficient to enable imaging of binding or complexing of the molecular probe to the EDB-FN and/or EDA-FN of the cancer cells or other cells in the cancer cell microenvironment.

The molecular probe comprising the targeting peptide described herein can be administered to the subject by, for example, systemic, topical, and/or parenteral methods of administration. These methods include, e.g., injection, infusion, deposition, implantation, or topical administration, or any other method of administration where access to the tissue by the molecular probe is desired. In one example, administration of the molecular probe can be by intravenous injection of the molecular probe in the subject. Single or multiple administrations of the probe can be given. “Administered”, as used herein, means provision or delivery of a molecular probe in an amount(s) and for a period of time(s) effective to label cancer cells in the subject.

Molecular probes comprising the targeting peptides described herein can be administered to a subject in a detectable quantity of a pharmaceutical composition containing a molecular probe or a pharmaceutically acceptable water-soluble salt thereof, to a patient.

A “detectable quantity” means that the amount of the molecular probe that is administered is sufficient to enable detection of binding or complexing of the probe to EDB-FN and/or EDA-FN expressed by the cancer cells or other cells in the cancer cell microenvironment. An “imaging effective quantity” means that the amount of the molecular probe that is administered is sufficient to enable imaging of binding or complexing of the molecular probe to the EDB-FN and/or EDA-FN of the cancer cells or other cells in the cancer cell microenvironment.

Formulation of the molecular probe to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like.

The preparation of a pharmacological composition that contains active ingredients dissolved or dispersed therein is well understood in the art. Typically such compositions are prepared as injectables either as liquid solutions or suspensions, however, solid forms suitable for solution, or suspensions, in liquid prior to use can also be prepared. Formulation will vary according to the route of administration selected (e.g., solution, emulsion, capsule).

Any polypeptide or compound may also be used in the form of a pharmaceutically acceptable salt. Acids, which are capable of forming salts with the polypeptides, include inorganic acids such as trifluoroacetic acid (TFA) hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like.

Bases capable of forming salts with the polypeptides include inorganic bases such as sodium hydroxide, ammonium hydroxide, potassium hydroxide and the like; and organic bases such as mono-, di- and tri alkyl and aryl-amines (e.g., tricthylamine, diisopropylamine, methylamine, dimethylamine and the like) and optionally substituted ethanolamines (e.g., ethanolamine, diethanolamine and the like).

The following examples are included for purposes of illustration and are not intended to limit the scope of the invention.

### EXAMPLES

### Example 1: ZD2-S0456 Binds to EDB-FN In Vivo in Human HNC Xenografts and Human Triple Negative Breast Cancer (TNBC) Xenografts

The specific staining of ZD2-S0456 to human HNC tumors comparing to normal tissues was investigated after intravenous injection of ZD2-S0456 in mouse xenograft models (FIG. 3). Mice were injected with 10 nmol/mouse ZD2-S0456 or PBS and euthanized 60 minutes post injection to collect tumor tissue and major organs. Fluorescence imaging revealed strong signal in the tumor (6) after injection of ZD2-S0456, and no signal after injection of PBS. There was fluorescence signal observed in the kidney, brain, and liver as well, but this was determined to be autofluorescence based on the results of the PBS injection. Furthermore, ZD2-S0456 can identify the tumor margin by topical staining of mouse xenograft tumors. The skin atop the HSC3 HNC xenograft tumor was removed to expose the tumor while the mouse was under anesthesia. Then 1 drop of 40 μM ZD2-Cy5.5 was topically applied to the tumor and surrounding tissues, which was rinsed with PBS after 2 minutes. Fluorescence images reveal strong fluorescence signal on the tumor but not the surrounding tissues (FIG. 3). These results indicate that ZD2 targeted fluorescence dyes are promising targeted imaging agents for visualizing HNC for intraoperative fluorescence imaging.

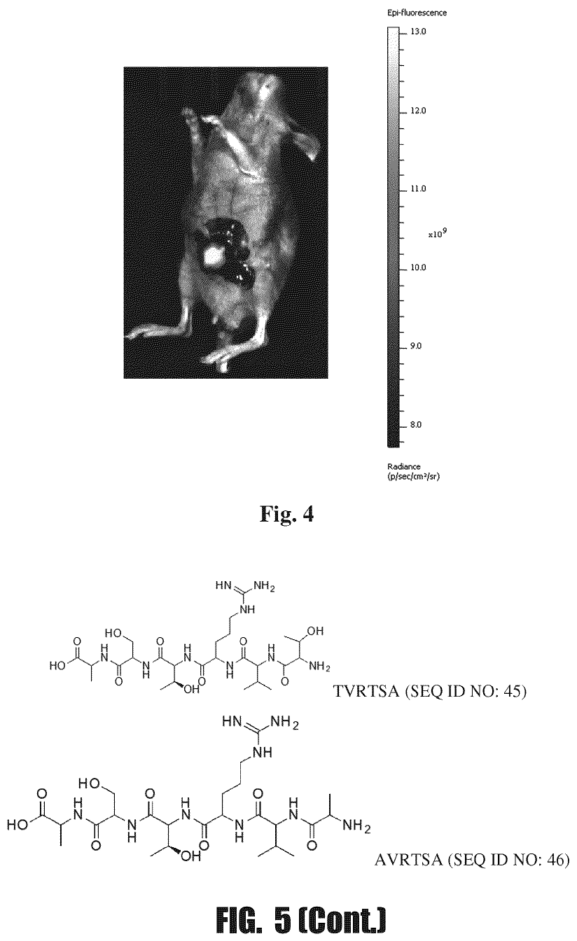

The specific staining of ZD2-S0456 to human TNBC tumors comparing to normal tissues was investigated after intravenous injection of ZD2-S0456 in mouse xenograft models (FIG. 4). Mouse were injected with 10 nmol/mouse ZD2-S0456 and imaged 60 minutes post injection. Fluorescence imaging revealed stronger signal in the tumor after injection of ZD2-S0456 comparing to surrounding normal tissues.

### Example 2: Synthesis and Examples of Molecular Probes

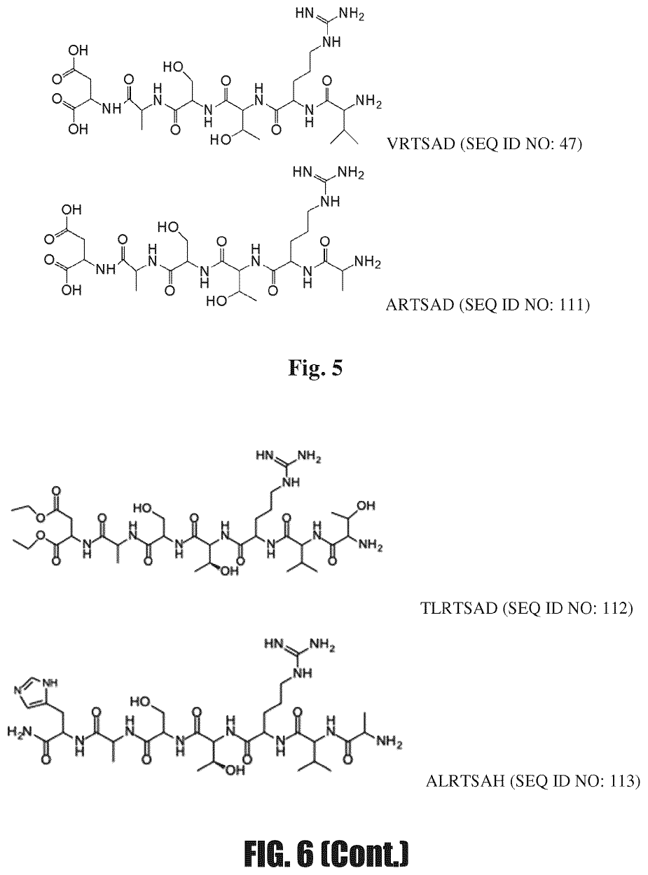

The inventors have carried out and proposed a number of synthetic procedures to provide the molecular probes described herein. FIG. 5 provides representative peptide sequences including deletion or truncation that can be used as targeting peptides in molecular probes, while FIG. 6 provides representative peptide sequences including C-terminal modification. The structure and amino acid makeup of the peptides is shown.

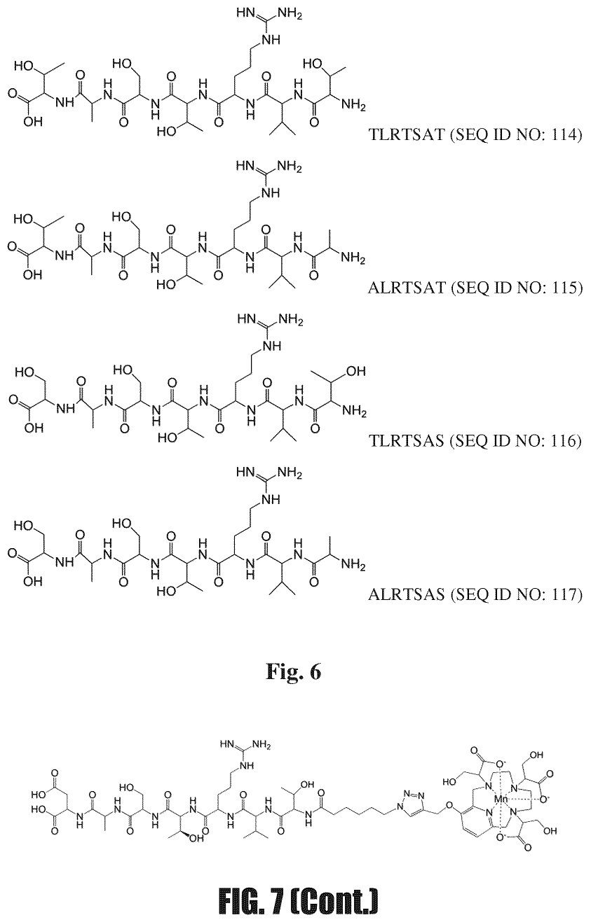

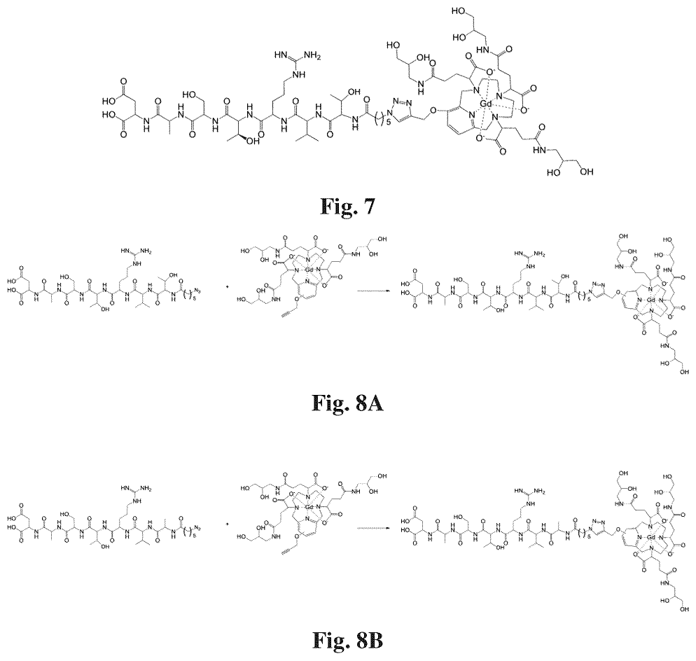

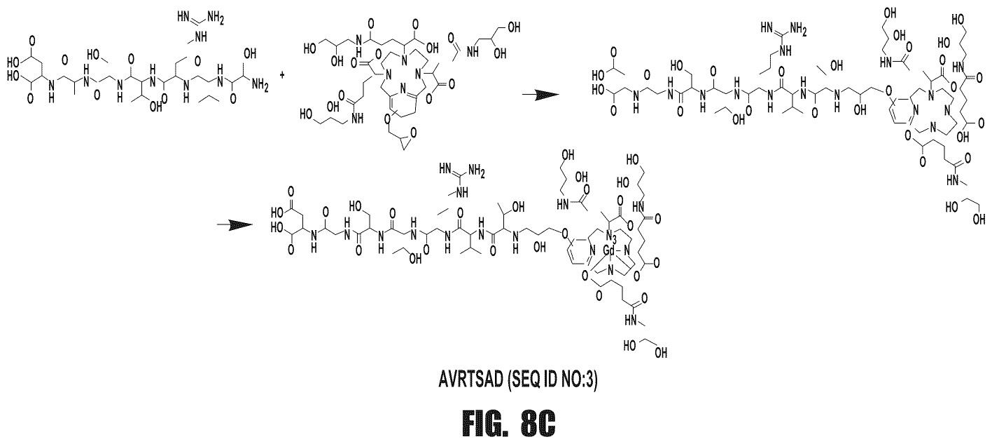

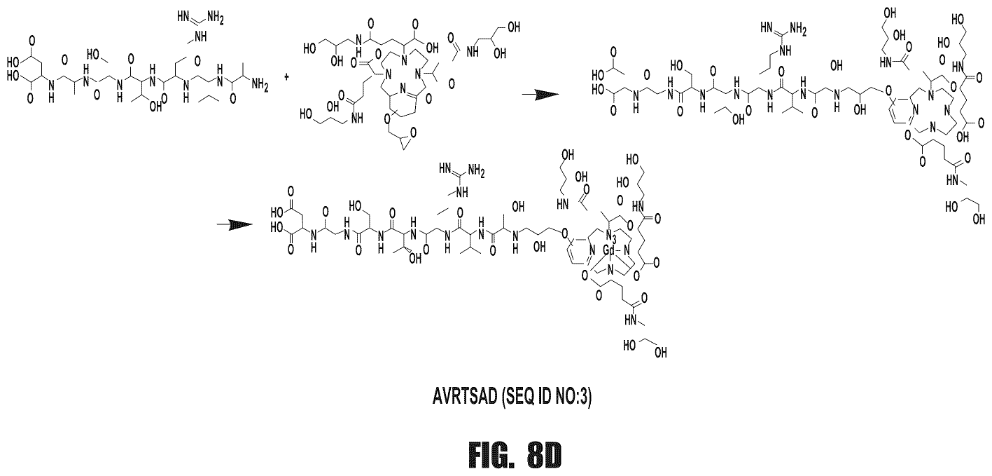

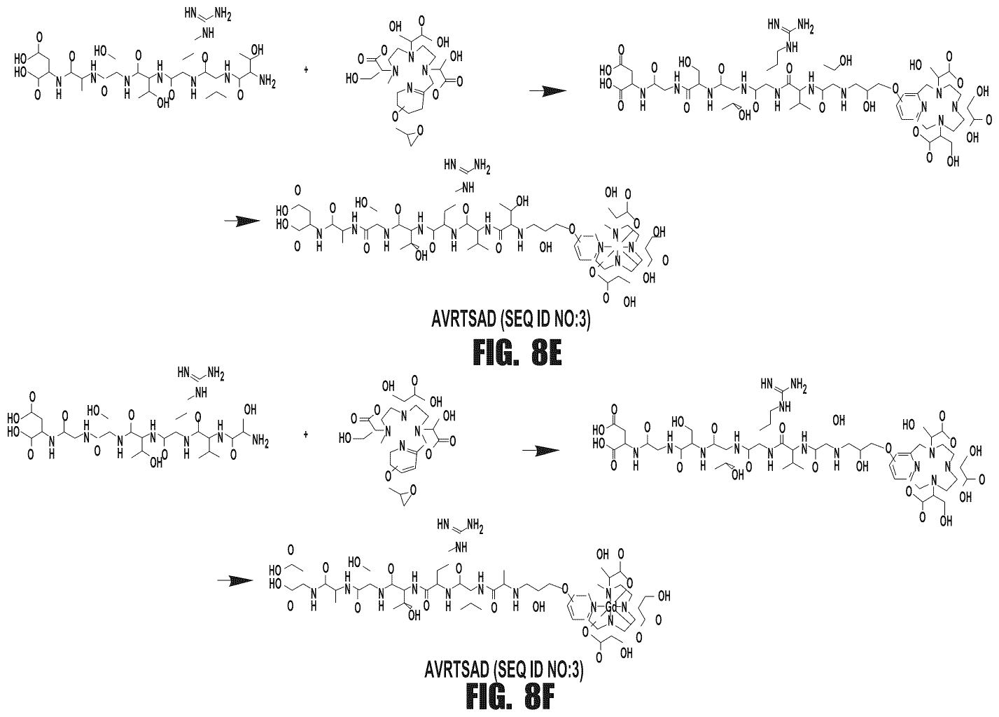

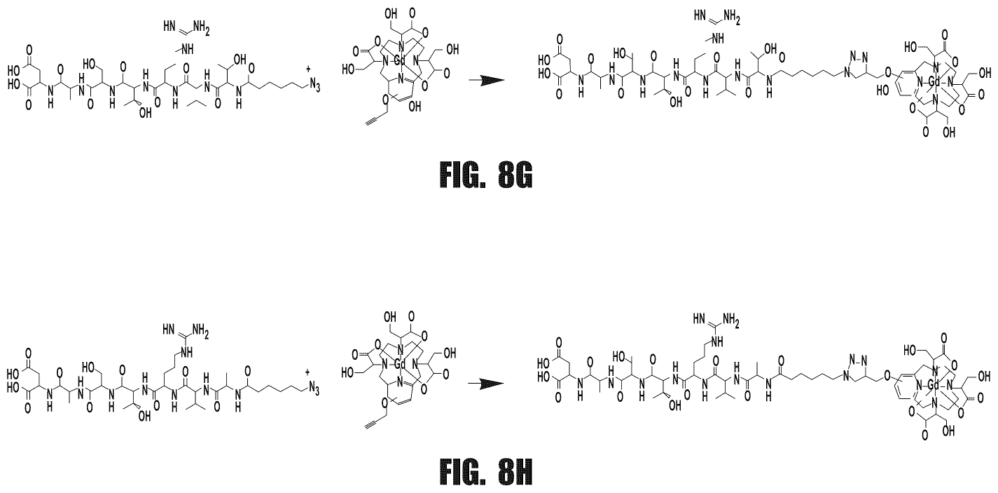

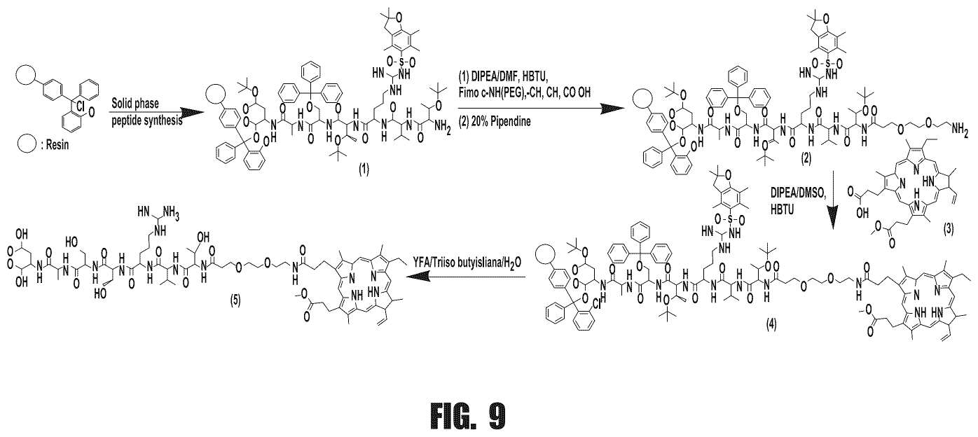

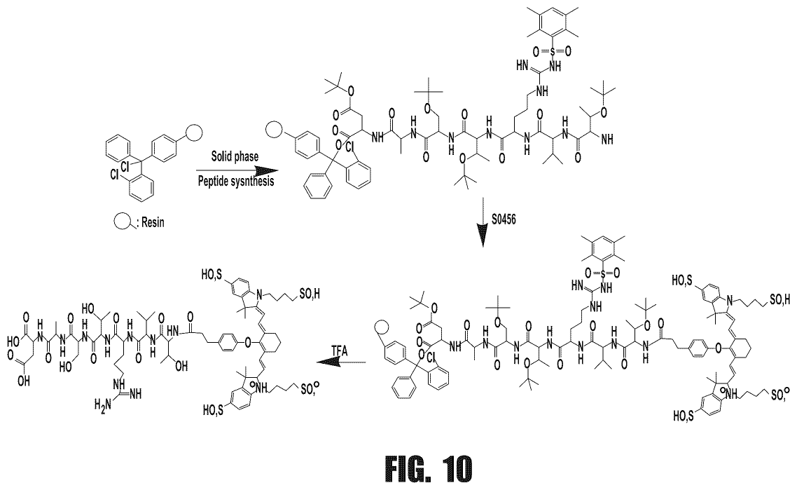

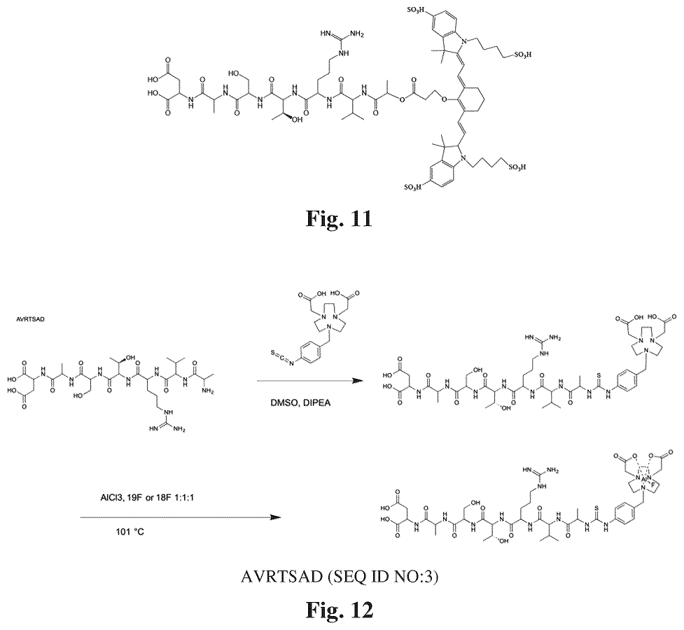

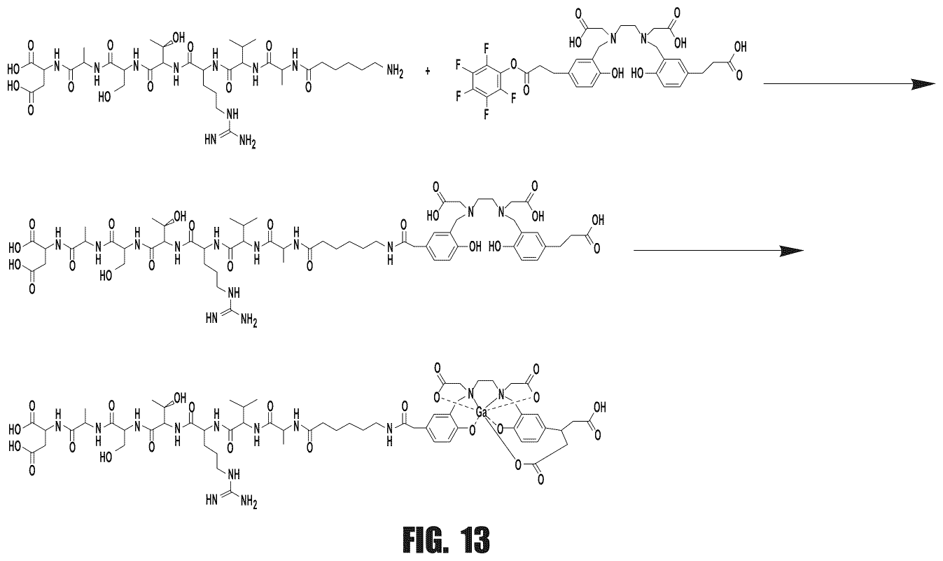

FIGS. 7-13 provide examples and synthetic pathways for various types of targeted contrast and imaging agents. FIG. 7 provides representative molecular probes including contrast agents. FIGS. 8A-8H provides various synthetic routes that can be used to prepare molecular probes including contrast agents. FIGS. 9-11 provide examples of NIR and fluorescent probes that can be used to image cancers or for imaging guided surgery. FIG. 9 provides a synthetic procedure for the near infrared imaging agent ZD2-PPIX. FIG. 10 provides a synthetic procedure for the fluorescent imaging agent ZD2-S0456. FIG. 11 provides the structure of the fluorescent imaging agent AVRTSAD-S0456. FIGS. 12 and 13 provide procedures for synthesizing PET imaging agents that can be used for detecting cancer. FIG. 12 provides a synthetic procedure for the PET imaging agent AVRTSAD-18F. Finally, FIG. 13 provides a synthetic procedure for the PET imaging agent AVRTSAD-HBEDCC-68Ga.

### Example 3: Detailed Synthesis of ZD2-PC3HMA

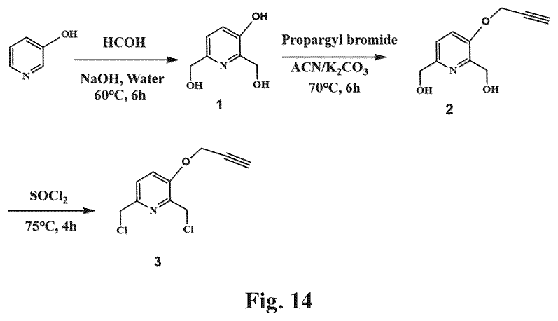

FIG. 14 shows the synthesis of compounds C1, C2, and C3. 3-Hydroxypyridine (11 mmol) was mixed with 37% formaldehyde (45 mmol) and sodium hydroxide (40 mmol) at 60° C. and stirred for 6 h. After reaction, removing formaldehyde and some water under vacuum, hydrochloric acid was added to the rest of solution to adjust pH=7. The C1 compound was purified by crystallization in water/methanol. The compound C1 (10 mmol) in water/acetonitrile (50/50) was mixed with propargyl bromide (15 mmol) in the presence of potassium carbonate (pH=9). The mixture was stirred at 70° C. 6 hours. The next day, after reaction, the product compound C2 was purified by C18 flash column. Compound C2 (20 mmol) was slowly mixed with thionyl chloride (60 mmol) and stirred at 70-80° C. for 4 hours. After cooling to room temperature, excess SOCl₂ was removed by evaporation, and then K₂CO₃ water solution was added. The solution was adjusted to pH 7 using K₂CO₃ aqueous solution. The product compound C3 was purified by HPLC and confirmed by ESI mass spectrum, m/z 230 (calculated M+1 230).

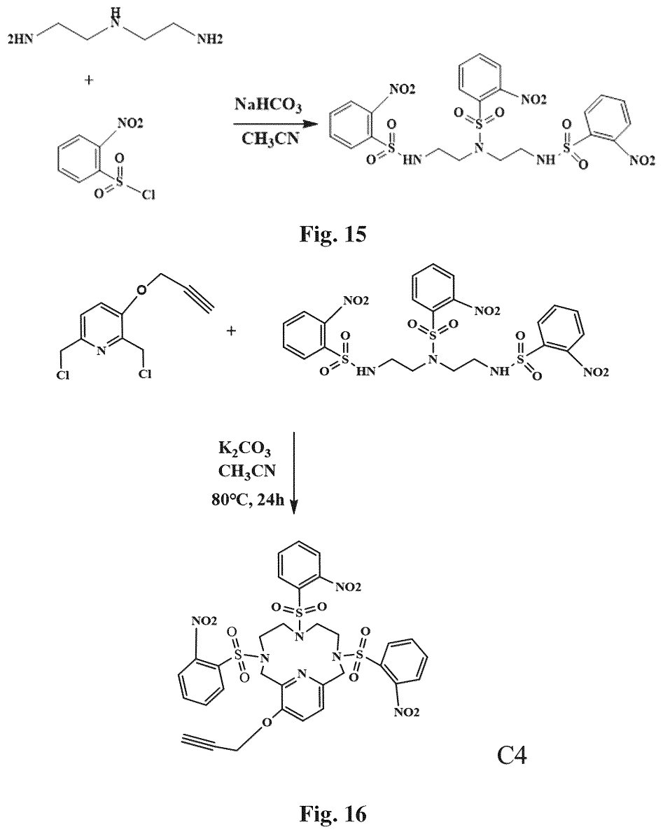

FIG. 15 shows the synthesis of tris(2-nitrobenzenesulfonyl) diethylenetriamine. The tris(2-nitrobenzenesulfonyl)-protected compound was prepared by reaction of 2-nitrobenzenesulfonyl chloride (10 mmol) in acetonitrile with diethylenetriamine (10 mmol) in the presence of sodium bicarbonate. The mixture was stirred at r.t. overnight. The product tris(2-nitrobenzenesulfonyl) diethylenetriamine was purified by flash chromatography, and confirmed by ESI mass spectrum, m/z 659 (M+1, calcd. 659.05), 681 (M+Na, calcd. 681.05), and 697 (M+K, calcd. 697.05).

FIG. 16 shows the synthesis of compound C4. The compound C3 (2 mmol) in acetonitrile mixed with the tris(2-nitrobenzenesulfonyl) diethylenetriamine (2 mmol) in the presence of potassium carbonate. The mixture was stirred at 80° C. for 1-day to give the macrocyclic compound C4. The product was purified by HPLC, and confirmed by ESI mass spectrum, m/z 816 (M+1, calcd 816.1), 838 (M+Na, calcd 838.1), and 854 (M+K, calcd 854.1).

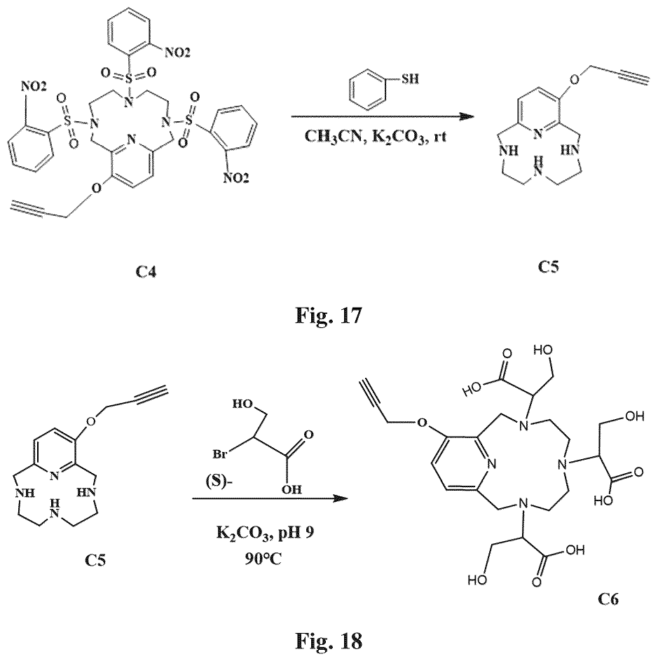

FIG. 17 shows the synthesis of compound 3,6,9,15-(12-propargyloxy) tetraazabicyclo [9.3.1]pentadeca-1 (15), 11,13-triene or PO-pyclen, C5. Compound C4 (0.5 mmol) in acetonitrile was readily deprotected with thiophenol (2 mmol) at room temperature in the presence of potassium carbonate water solution to produce PO-pyclen C5. The mixture was stirred at r.t. overnight. The product C5 was purified by HPLC, and confirmed by ESI mass spectrum, m/z 261 (M+1, calcd. 261.34), and 283 (M+Na, calcd. 283.34).

FIG. 18 shows the synthesis of compound 3,6,9,15-(12-propargyloxy) tetraazabicyclo [9.3.1]pentadeca-1 (15), 11,13-triene-3,6,9,-tri (2′-hydroxymethylacetic acid) or PC3HMA, C6. (S)-2-Bromo-3-hydroxypropanoic acid (20 mmol) dissolved in water was drop-wisely added to the compound C5 (5 mmol) water solution in the presence of potassium carbonate at 90° C. Then the potassium carbonate solution was added dropwise to the mixture with stirring at 90° C. The reaction mixture was stirred overnight at 90° C. The product compound C6 was purified by HPLC, and confirmed by ESI mass spectrum, m/z 525 (M+1, calcd. 525.2), and 547 (M+Na, calcd. 547.2).

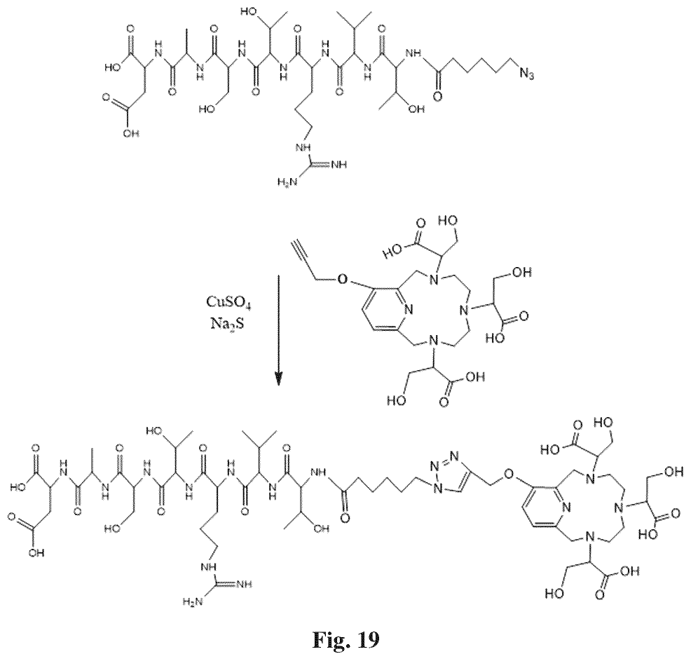

FIG. 19 shows the synthesis of ZD2-PC3HMA. To aqueous solution of alkynyl-Pyclen-PR (0.13 mmol) (compound C6) and ZD2-C6-N₃ (0.2325 mmol), CuSO₄·5H₂O (0.3 mmol) and ascorbic acid (1.32 mmol) were added. The pH of the solution was adjusted to 8˜9 by 0.1 N NaOH, and the mixture was stirred at room temperature for 24 h. After reaction sodium sulfide was added to the mixture solution to precipitate copper ions. The product ZD2-PC3HMA was purified by HPLC and confirmed by ESI mass spectrum, m/z 1412.53 (M+1, calcd. 1412.65).

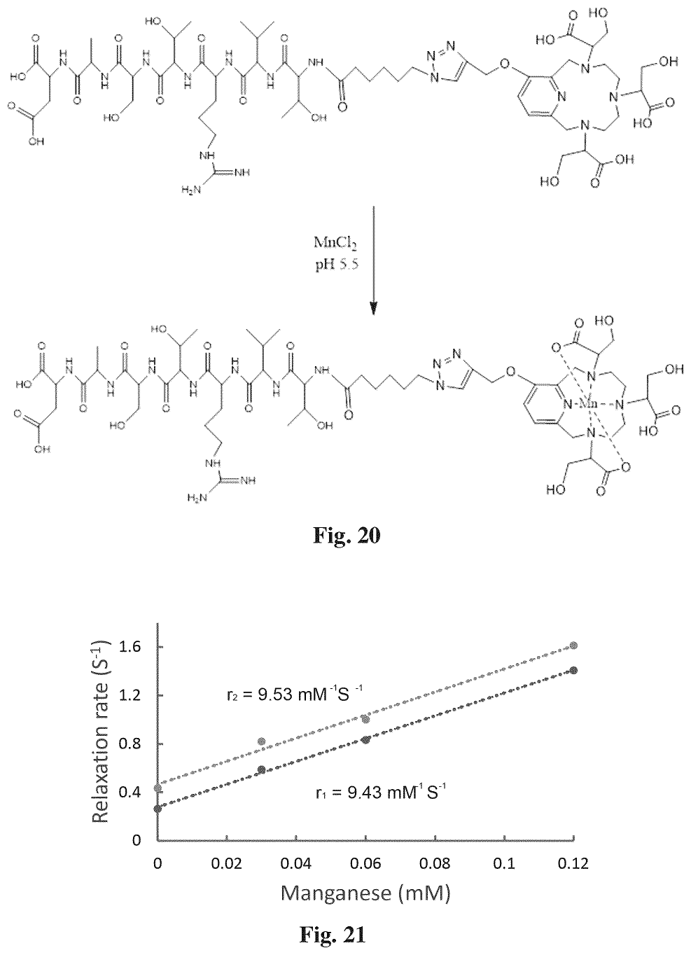

FIG. 20 shows the synthesis of ZD2-(Mn-PC3HMA). Manganese chloride (0.15 mmol) in water was added to the ZD2-PC3HMA) (0.1 mmol) water solution, and pH was adjusted to 5.5. The mixture was stirred for 8 hours. The product ZD2-(Mn-PC3HMA) was purified by HPLC, and confirmed by ESI mass spectrum, m/z 1465.40 (M+1, calcd. 1465.58), and 1487.40 (M+Na, calcd. 1487.58).

FIG. 21 shows the relaxivity for ZD2-(Mn-PC3HMA). The T₁ and T₂ relaxation times of ZD2-(Mn-PC3HMA) in PBS at 1.4 T were measured using a Bruker Minispec Relaxometer (60 Hz, 37° C.; Bruker, Billerica, MA, USA). The T₁ and T₂ relaxivities were calculated as the slope of the 1/T₁ and 1/T₂ vs Mn concentration, respectively. Relaxivities of ZD2-(Mn-PC3HMA) in PBS are r₁=9.43 mM⁻¹S⁻¹ and r₂=9.53 mM⁻¹S⁻¹.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A molecular probe compound according to the following formula:
P-L-I
wherein P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof.

2. The molecular probe of claim 1, wherein L comprises at least one of polyalkyleneoxide, polyvinyl alcohol, polyethylene glycol (PEG), polypropylene glycol (PPG), co-poly (ethylene/propylene) glycol, polyoxyethylene (POE), polyurethane, polyphosphazene, polysaccharide, dextran, polyvinylpyrrolidone, polyvinyl ethyl ether, polyacryl amide, polyacrylate, or polycyanoacrylate.

3. The molecular probe of claim 1, wherein the imaging compound is according to formula I: 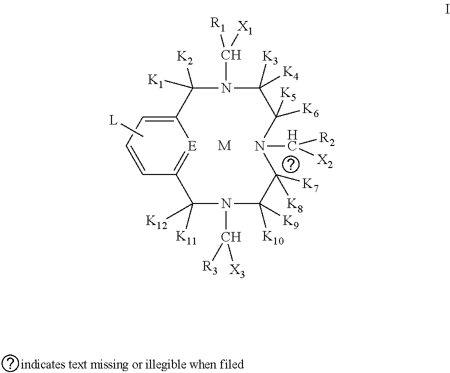
wherein: R₁, R₂ and R₃ represent, independently of one another, —COOH, —P(O)(OH)₂ or —R₆—P(O)—OH in which R₆ represents an H atom or a C₁-C₃ alkyl group, preferably COOH; X₁, X₂ and X₃ represent, independently of one another, —(CH₂)ₐOH_{b} with a,b=1 to 3, advantageously —CH₂OH, —CH₂—CH₂OH, —CHOH—CH₂OH, —CH—(CH₂OH)₂, or L-Y in which L represents a C1-C3 alkyl group, preferably (CH₂) n with n=1 to 3, Y represents CO—NR₇R₈, in which R₇ represents H or a C1-C6 alkyl group or a C1-C6 hydroxyalkyl group, in particular a C2-C4 group, advantageously —CH₂—CH₂OH, —CHOH—CH₂OH, —CH—(CH₂OH)₂, —(CH₂)ₘ—(CHOH)ₚ—CH₂OH, with m=1 to 3, p=1 to 4 and m+p=2 to 5, or —C—(CH₂OH)₃, and R₈ represents a C1-C6 alkyl or C1-C6 hydroxyalkyl group, in particular a C2-C4 group, advantageously —CH₂—CH₂OH, —CHOH—CH₂OH, —CH—(CH₂OH)₂, —(CH₂)ₘ—(CHOH)ₚ—CH₂OH, with m=1 to 3, p=1 to 4 and m+p=2 to 5, or —C—(CH₂OH)₃, provided that at least R₇ or R₈ represents a C1-C6 hydroxyalkyl group; D represents CH or N; E represents CH or N; F₁ represents CH or N; K₁ to K₁₂—each independently represent H, —(CH₂)j-CH₃ or —(CH₂)i-OH, in which j=0 to 3 and i=1 to 3, advantageously H, or K₃ or K₄ with K₅ or K₆, and/or K₇ or K₈ with K₉ or K₁₀, form a ring having 3 to 6 carbon atoms; and M is a paramagnetic ion selected from Gd⁺³, Eu⁺³, Tm⁺³, Dy⁺³, Yb⁺³, Mn⁺², and Fe⁺³.

4. The molecular probe according to claim 3, wherein K₁-K₁₂ are H.

5. The molecular probe according to claim 3, wherein D is C, E is N, and F1 is CH.

6. The molecular probe according to claim 3, wherein R₁, R₂, and R₃ are —COOH.

7. The molecular probe according to claim 3, wherein X₁, X₂, and X₃ are —CH₂OH.

8. The molecular probe according to claim 3, wherein M is Mn²⁺ or Gd³⁺.

9. The molecular probe according to claim 1, wherein the targeting peptide comprises SEQ ID NO: 1 or SEQ ID NO: 2.

10. The molecular probe according to claim 1, wherein the imaging agent is a fluorescent imaging agent.

11. The molecular probe according to claim 10, wherein the imaging agent is according to formula II:

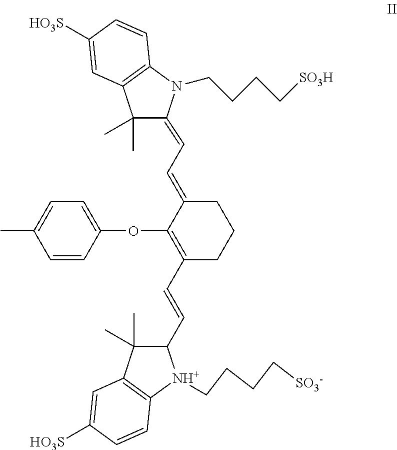

12. The molecular probe according to claim 1, wherein the imaging agent is a PET imaging agent.

13. The molecular probe according to claim 1, wherein the imaging agent is a near infrared imaging agent.

14. A method of detecting cancer in a subject, comprising contacting a tissue of the subject with an effective amount of a molecular probe comprising the formula
P-L-I,
wherein P is a targeting peptide selected from the group consisting of SEQ ID NO: 1 to SEQ ID NO: 117; I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof;
detecting the amount of the molecular probe present in the tissue;
comparing the amount of molecular probe detected to a control value; and
detecting cancer in the subject if the amount of the molecular probe present in the tissue is higher than the control value.

15. The method of claim 14, wherein the cancer is breast cancer, oral cancer, pancreatic cancer, or prostate cancer.

16. The method of claim 14, wherein the molecular probe comprises a contrast agent.

17. The method of claim 16, wherein the contrast agent includes a paramagnetic ion selected from the group consisting of Gd⁺³, Eu⁺³, Tm⁺³, Dy⁺³, Yb⁺³, Mn⁺², and Fe⁺³; and salts thereof.

18. The method of claim 14, wherein the molecular probe comprises a near infrared imaging agent.

19. The method of claim 14, wherein the molecular probe comprises a fluorescent imaging agent.

20. The method of claim 14, wherein the molecular probe comprises a PET imaging agent.

21. The method of claim 14, wherein the tissue is contacted in vivo.

22. The method of claim 14, wherein the molecular probe is systemically administered to a subject having or suspected of having cancer.

23. A method of monitoring the treatment of cancer in a subject, comprising:
contacting a tissue of a subject undergoing treatment of cancer with an effective amount of a molecular probe comprising the formula
P-L-I
for a first time, wherein:
P is a targeting peptide selected from the group consisting of SEQ ID NO: 1 to SEQ ID NO: 117;
I is an imaging agent; and L is an optional non-peptide linker that covalently links the peptide to the imaging agent, wherein the linker comprises a carboxylic acid that forms a carboxamide with an amine of the targeting peptide or a maleimide that forms a thioester bond with a cysteine residue of the targeting peptide, or a pharmaceutically acceptable salt thereof;
detecting a first amount of the molecular probe present in the tissue;
contacting the tissue of the subject for a second time with an effective amount of the molecular probe comprising the formula P-L-I;
detecting a second amount of the molecular probe present in the tissue; and
comparing the first amount and the second amount of the molecular probe to monitor the treatment of cancer in the subject.

24. The method of claim 23, wherein the cancer is breast cancer, oral cancer, pancreatic cancer, or prostate cancer.

25. The method of claim 23, wherein the tissue is contacted in vivo.

26. The method of claim 23, wherein the cancer is being treated with a chemotherapeutic agent.

27. The method of claim 23, wherein the molecular probe comprises a contrast agent.

28. The method of claim 27, wherein the contrast agent includes a paramagnetic ion selected from the group consisting of Gd⁺³, Eu⁺³, Tm⁺³, Dy⁺³, Yb⁺³, Mn⁺², Fe⁺³; and salts thereof.

29. The method of claim 23, wherein the molecular probe comprises a near infrared imaging agent.

30. The method of claim 23, wherein the molecular probe comprises a fluorescent imaging agent.

31. The method of claim 23, wherein the molecular probe comprises a PET imaging agent.
